(19) United States
(12) Patent Application Publication
(10) Pub. No.: US 2025/0114729 A1
(43) Pub. Date: Apr. 10, 2025

(54) SEPARATION APPARATUS AND SEPARATION METHOD

(71) Applicants: CANON KABUSHIKI KAISHA, Tokyo (JP); Canon Medical Systems Corporation, Otawara-shi (JP)

(72) Inventors: Yuhi HIGUCHI, Tokyo (JP); Tsutomu MIKI, Komae (JP)

(73) Assignees: CANON KABUSHIKI KAISHA, Tokyo (JP); Canon Medical Systems Corporation, Otawara-shi (JP)

(21) Appl. No.: 18/904,835

(22) Filed: Oct. 2, 2024

(30) Foreign Application Priority Data
Oct. 6, 2023 (JP) 2023-174694

(51) Int. Cl. B01D 29/66; A61M 1/36; B01D 29/01; B01D 29/90; B01D 29/96

(52) U.S. Cl. CPC B01D 29/668; A61M 1/3692; B01D 29/01; B01D 29/90; B01D 29/96; B01D 2201/167; B01D 2201/204

(57) ABSTRACT

According to one embodiment, a separation apparatus includes a filter configured to separate a white blood cell from blood sent from a blood container, and a first flow channel through which the blood container and the filter are in communication with each other. The separation apparatus introduces, through the first flow channel, the blood into the filter from below and in an anti-gravity direction.

### CROSS-REFERENCE TO RELATED APPLICATION(S)

This application is based upon and claims the benefit of priority from the prior Japanese Patent Application No. 2023-174694, filed Oct. 6, 2023, the entire contents of which are incorporated herein by reference.

### FIELD

Embodiments described herein relate generally to a separation apparatus and a separation method.

### BACKGROUND

A cell production process for producing cells includes, for example, a step of separating cells from donor tissue and a step of culturing the separated cells. For application to the cell separating step, for example, a method is known which sets a filter at a part of the piping for the blood of a donor to flow and separates and collects white blood cells from the blood flowing through the piping. In one example, this method proceeds in such a manner that the white blood cells are separated by the filter from the blood flowing in the gravity direction, the filter is washed with a rinse liquid flowing in the gravity direction, and the white blood cells are collected from the filter using the rinse liquid flowing in the anti-gravity direction. In instances where the method is adopted with an open-system flow channel, the blood introduced into, for example, a disk-shaped filter easily spreads to every part of the filter, and thus, the blood can fill the filter without allowing air bubbles to be left in the filter.

On the other hand, in instances where the method is adopted with a closed-system flow channel, the blood introduced into a disk-shaped filter tends to initially move in a single direction and then take a circular motion inside the filter, and thus, air bubbles are often left in the filter and the blood can only fill the space in the filter where the air bubbles are not present. Here, the state of the filter in which air bubbles are left is not desirable since it could permit red blood cells to remain behind the air bubbles even after washing of the filter, and such remaining red blood cells could be collected together with the white blood cells.

### BRIEF DESCRIPTION OF THE DRAWINGS

FIG. 1 is a block diagram showing an exemplary configuration of a separation apparatus according to a first embodiment.

FIG. 2 is a schematic diagram for explaining one example of the filter shown in FIG. 1.

FIG. 3 is a schematic diagram for explaining another example of the filter shown in FIG. 1.

FIG. 4 is a flowchart for explaining an exemplary operation in the first embodiment.

FIG. 5 is a schematic diagram for explaining an operation in the first embodiment.

FIG. 6 is a schematic diagram for explaining a length of the first flow channel shown in FIG. 5.

FIG. 7 is a schematic diagram for explaining an operation in the first embodiment.

FIG. 8 is a schematic diagram for explaining an operation in the first embodiment.

FIG. 9 is a schematic diagram for explaining an operation in the first embodiment.

FIG. 10 is a block diagram showing an exemplary configuration of a separation apparatus according to Modification 1 of the first embodiment.

FIG. 11 is a schematic diagram for explaining an operation in Modification 1 of the first embodiment.

FIG. 12 is a schematic diagram for explaining an operation in Modification 1 of the first embodiment.

FIG. 13 is a diagram for explaining a holder of a filter according to Modification 2 of the first embodiment.

FIG. 14 is a schematic diagram for explaining flipping over of the holder shown in FIG. 13.

FIG. 15 is a block diagram showing an exemplary configuration of a separation apparatus according to a second embodiment.

FIG. 16 is a block diagram showing an exemplary configuration of a separation apparatus according to Modification 1 of the second embodiment.

FIG. 17 is a block diagram showing an exemplary configuration of a separation apparatus according to Modification 2 of the second embodiment.

FIG. 18 is a block diagram showing an exemplary configuration of a separation apparatus according to a third embodiment.

FIG. 19 is a flowchart for explaining an exemplary operation in the third embodiment.

FIG. 20 is a schematic diagram for explaining an operation in the third embodiment.

FIG. 21 is a schematic diagram for explaining an operation in the third embodiment.

FIG. 22 is a block diagram showing an exemplary configuration of a separation apparatus according to Modification 1 of the third embodiment.

FIG. 23 is a schematic diagram for explaining an operation in Modification 1 of the third embodiment.

FIG. 24 is a schematic diagram for explaining an operation in Modification 1 of the third embodiment.

FIG. 25 is a block diagram showing an exemplary configuration of a separation apparatus according to a fourth embodiment.

FIG. 26 is a flowchart for explaining an exemplary operation in the fourth embodiment.

FIG. 27 is a schematic diagram for explaining an operation in the fourth embodiment.

FIG. 28 is a schematic diagram for explaining an operation in the fourth embodiment.

### DETAILED DESCRIPTION

In general, according to one embodiment, a separation apparatus includes a filter configured to separate a white blood cell from blood sent from a blood container, and a first flow channel through which the blood container and the filter are in communication with each other. The separation apparatus introduces, through the first flow channel, the blood into the filter from below and in an anti-gravity direction.

The separation apparatus and the separation method according to each embodiment will be described with reference to the drawings. Note that the embodiments and the modifications described below are each workable in combination with other one or more embodiments and/or modifications. By way of example, the description will assume instances where white blood cells for use in production of induced pluripotent stem (iPS) cells are separated by the separation apparatus. However, the separation apparatus and the separation method are not limited to the production of iPS cells but may be applicable to any uses involving the separation of white blood cells.

### First Embodiment

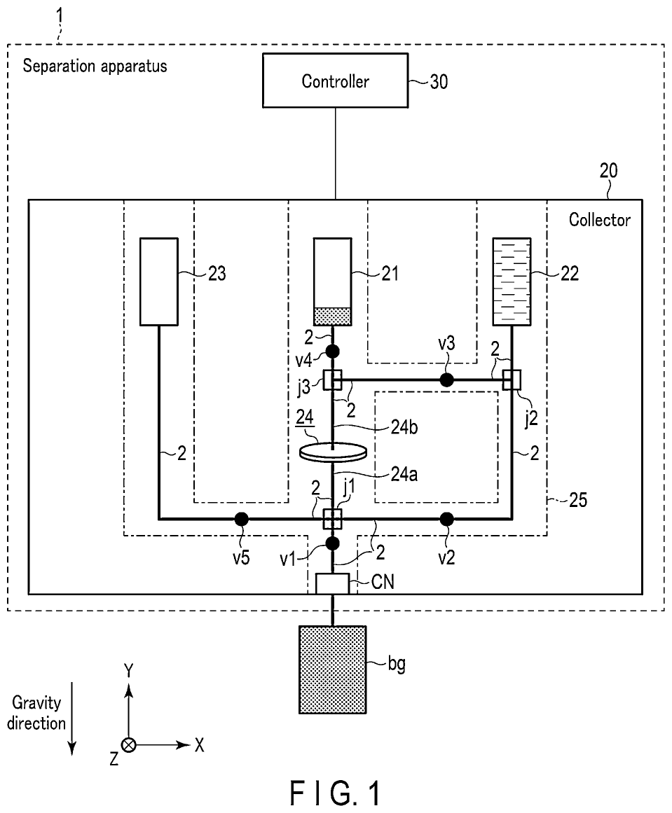

FIG. 1 is a block diagram showing an exemplary configuration of the separation apparatus according to the first embodiment. This separation apparatus, denoted by reference sign “1”, includes a collector 20 and a controller 30.

The collector 20 here is implemented in the form of, for example, a cartridge adapted to be detachably held by the separation apparatus 1. The collector 20 includes substantially-closed-system flow channels and a substantially-closed-system container which accommodates the flow channels. The substantially-closed-system flow channels include a drain container 21, a medical liquid container 22, a collection container 23, a filter 24, multiple pipes 2, joints j1 to j3, valves v1 to v5, and a sterile connector CN. The substantially-closed-system flow channels have a first closed architecture and are held by a retainer 25 which is formed along the flow channels. This retainer 25 may be constituted by multiple guide plates or an accommodating member embracing all of the flow channels. The retainer 25 functions as a substantially-closed-system container and has a second closed architecture outside the flow channels of the first closed architecture so that a liquid does not leak out from the collector 20. The collector 20 therefore has a double-closed architecture constituted by the first closed architecture and the second closed architecture. However, the collector 20 is not limited to such a double-closed architecture but may adopt a triple-closed architecture which further has a container enclosing the double-closed architecture so as to enhance the safety even more. The triple-closed architecture is not a limitation, either, and the collector 20 may instead adopt a multi-closed architecture which uses four or more closed systems. In one example, the body of the collector 20 and the retainer 25 are rigid or hard and made of a resin or plastic material. The resin material is, for example, polycarbonate (PC) or polystyrene (PS). Preferably, the body of the collector 20 has a light transmitting property. It is preferable that the body of the collector 20 be light transmitting because, for example, it allows for easy observation of the white blood cell collecting process.

The sterile connector CN is freely attachable to and detachable from the pipe 2 for a blood container bg which is assumed to be an externally provided blood bag here. The joint j1 has a substantially crisscross shape with branching portions, and is connected to four pipes 2 so that these four pipes 2 are in communication with one another. More specifically, the joint j1 places four pipes 2 which are respectively connected to the joint j2, the collection container 23, the filter 24, and the sterile connector CN, in a mutually communicating state. The joints j2 and j3 each have a substantially T shape with branching portions, and they are each connected to three pipes 2 so that these three pipes 2 are in communication with one another. More specifically, the joint j2 places three pipes 2 which are respectively connected to the joint j1, the medical liquid container 22, and the joint j3, in a mutually communicating state. The joint j3 places three pipes 2 which are respectively connected to the joint j2, the filter 24, and the drain container 21, in a mutually communicating state. The pipes 2 are provided with the valves v1 to v5 as appropriate. In one example, the pipes 2 are elastically deformable members, and the valves v1 to v5 close the respectively corresponding pipes 2 by elastically deforming and squashing them and open them by releasing and restoring them. The valve v1 is disposed at the pipe 2 that connects the joint j1 and the sterile connector CN to each other. The valve v2 is disposed at the pipe 2 that connects the joints j1 j2 to each other. The valve v3 is disposed at the pipe 2 that connects the joints j2 and j3 to each other. The valve v4 is disposed at the pipe 2 that connects the joint j3 and the drain container 21 to each other. The valve v5 is disposed at the pipe 2 that connects the joint j1 and the collection container 23. Each of these valves v1 to v5 may be, for example, a pinch valve adapted to be controlled by the controller 30 for opening and closing actions. Also, the pipes 2 are each constituted by, for example, an airtight flexible tubular member such as a vinyl or plastic tube. Each liquid flowing through the pipes 2 is thus inhibited or prevented from being exposed to the ambient air.

The collector 20 with these features can form various flow channels capable of being switched from one another by utilizing the opened states of the valves v1 to v5. For example, for separating white blood cells from blood, the valves v1 and v4 are placed in the opened state so that a flow channel is formed from the blood container bg containing the blood of a donor to the drain container 21 via the valve v1, the filter 24, and the valve v4. Also, for rinsing the filter 24, the valves v2 and v4 are placed in the opened state so that a flow channel is formed from the medical liquid container 22 to the drain container 21 via the valve v2, the filter 24, and the valve v4. For collecting white blood cells from the filter 24, the valves v3 and v5 are placed in the opened state so that a flow channel is formed from the medical liquid container 22 to the collection container 23 via the valve v3, the filter 24, and the valve v5.

Each of the drain container 21, the medical liquid container 22, and the collection container 23 is a container adapted for liquid accommodation and having a configuration capable of discharging and/or suctioning a liquid. One example of the configuration capable of discharging and/or suctioning a liquid is a syringe including a cylinder and a piston. Another example of the configuration is a substantially-closed-system container connected to a pumping mechanism which produces a positive and/or negative gas pressure. The embodiment will assume that each of the drain container 21, the medical liquid container 22, and the collection container 23 adopts a syringe.

The drain container 21 in one example is constituted by a syringe including a cylinder and a piston. The drain container 21 is adapted to contain, as a drained liquid, a liquid such as blood and/or a rinse liquid in its cylinder with an airtight configuration so that the liquid is inhibited or prevented from being exposed to the ambient air. The tip of the drain container 21 is connected to the joint j3 via the applicable pipe 2. The drain container 21 is controlled by the controller 30 to carry out a piston pulling action so as to take in the blood or the rinse liquid present in the pipe 2 through its tip. The drain container 21 as such is suitable for realizing quantitative and accurate liquid delivery.

The medical liquid container 22 in one example is constituted by a syringe including a cylinder and a piston. The medical liquid container 22 is adapted to contain a rinse liquid in its cylinder with an airtight configuration so that the rinse liquid is inhibited or prevented from being exposed to the ambient air. The tip of the medical liquid container 22 is connected to the joint j2 via the applicable pipe 2. The medical liquid container 22 is controlled by the controller 30 to carry out a piston pushing action so as to discharge the rinse liquid present in the cylinder through its tip. The rinse liquid is, for example, a phosphate buffer saline solution (PBS) or the like, and a liquid which imparts very little damage to white blood cells may be adopted as the rinse liquid. The medical liquid container 22 as such is suitable for realizing quantitative and accurate liquid delivery. The medical liquid container 22 is an example of a first medical liquid container and a second medical liquid container. The rinse liquid is used both as a wash liquid for washing the filter 24 and as a collection liquid for collecting white blood cells from the filter 24. The rinse liquid is an example of the wash liquid and the collection liquid in instances where these liquids are the same as each other.

The collection container 23 in one example is constituted by a syringe including a cylinder and a piston. The collection container 23 is adapted to contain a liquid such as a blood component and/or the rinse liquid including the collected white blood cells in its cylinder with an airtight configuration so that the liquid is inhibited or prevented from being exposed to the ambient air. The tip of the collection container 23 is connected to the joint j1 via the applicable pipe 2. The collection container 23 is controlled by the controller 30 to carry out a piston pulling action so as to take in the liquid present in the pipe 2 through its tip. The collection container 23 as such is suitable for realizing quantitative and accurate liquid delivery.

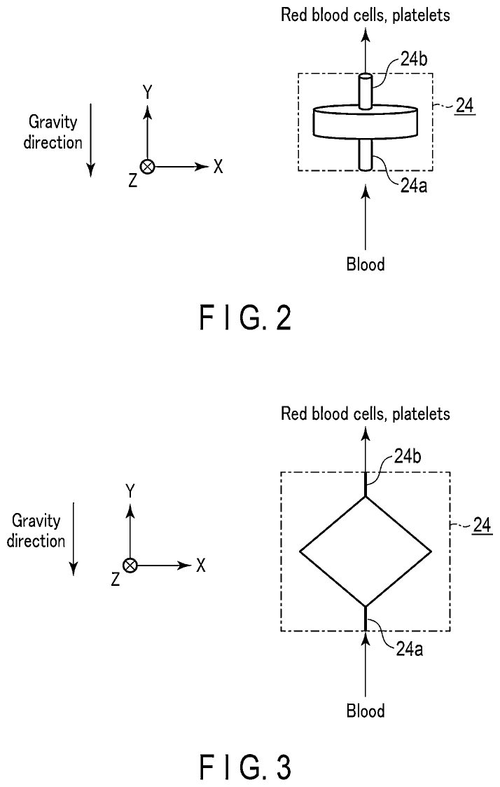

The filter 24 is disposed between the blood container bg and the drain container 21 and separates white blood cells from the blood sent from the blood container bg. In one example, the filter 24 is constituted by a material having meshes that can catch white blood cells in blood while permitting other substances (e.g., red blood cells and platelets) having a smaller particle size than the white blood cells to pass through, and a filter container accommodating the material. The material of the filter 24 is, for example, a nonwoven fabric. That is, each white blood cell as a target cell having a particle size larger than the opening of such meshes is caught by the filter 24. Red blood cells, etc., not handled as target cells in this example, each have a particle size smaller than the mesh opening, and therefore, they pass through the filter 24. The filter 24 is arranged so that, as shown in FIG. 2 or 3, an inlet 24a for introducing blood comes to a lower part and an outlet 24b for discharging blood components (e.g., red blood cells and platelets) remaining after the white blood cell separation comes to the upper part. The filter 24 may adopt any shape as appropriate, such as a disk shape as shown in FIG. 2 or a diamond shape as shown in FIG. 3. For example, as the filter 24, a white blood cell removing filter such as a product named Acrodisc WBC syringe filter (AP-4951) available from Pall Corporation, a product named Sepacell RZ (RZ-1000N, RZ-2000N, etc.) available from Asahi Kasei Medical Co., Ltd. etc., may be employed as appropriate. Note that the white blood cell removing filter here may instead be called a “red blood cell removing filter” according to the intended use or purpose. The embodiment will assume that the filter 24 is a disk-shaped filter as shown in FIG. 2.

The controller 30 is, in one example, processing circuitry such as a processor and takes total control over the separation apparatus 1. For example, the controller 30 controls opening and closing actions of valves v1 to v5 so as to switchably form various flow channels. For the formed flow channels, the controller 30 also controls discharging and suctioning actions of the drain container 21, the medical liquid container 22, and the collection container 23. An entire operation of the separation apparatus 1 may be called a “blood cell removing operation”.

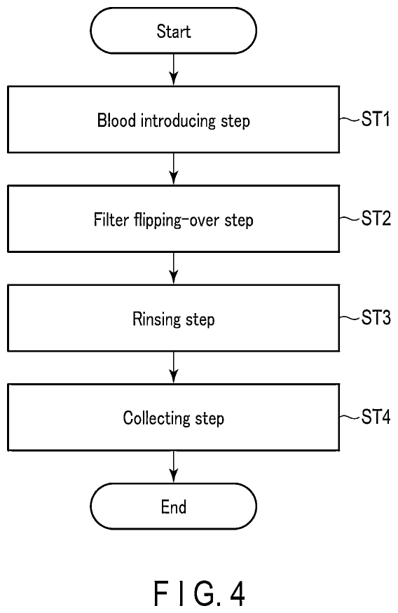

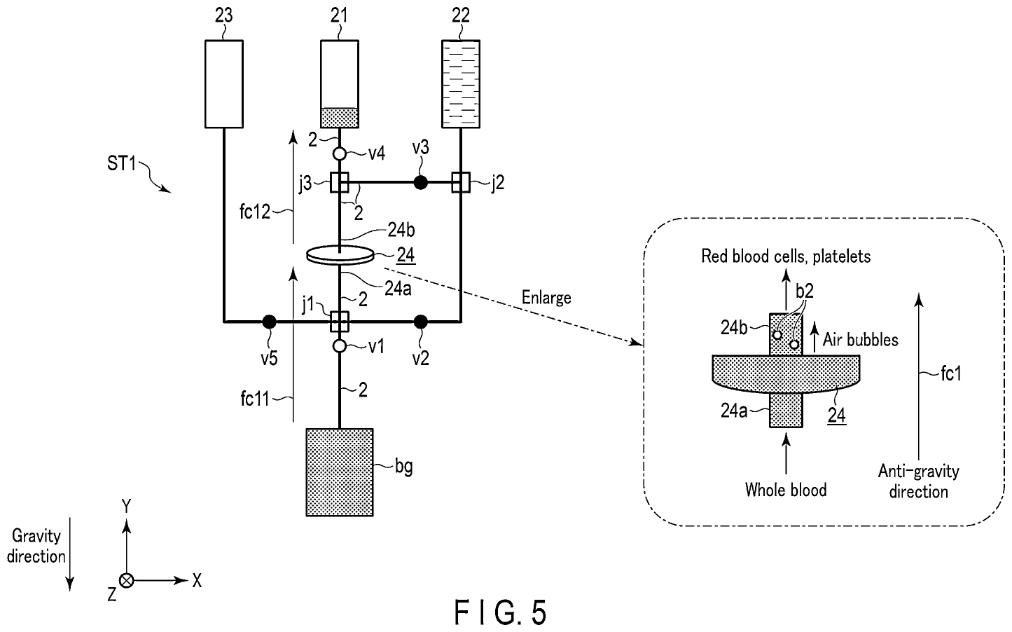

An exemplary operation of the separation apparatus 1 configured as above will be described with reference to the flowchart in FIG. 4 and the schematic diagrams in FIGS. 5 to 9. For the sake of explanation, it will be assumed that the valve v1 shown as a white circle mark indicates the opened-state valve v1 and the valve v1 shown as a black circle mark indicates the closed-state valve v1. It will likewise be assumed that the white circle marks for the valves v2 to v5 indicate these valves being in the opened state and the black circle marks for the valves v2 to v5 indicate these valves being in the closed state. This will also apply to each of the additional valves involved in the subsequent embodiments, etc.

The description will assume that the blood container bg is in a state of storing blood collected from a donor, and the medical liquid container 22 is in a state of storing a PBS (saline solution) serving as the rinse liquid. It will be assumed that the drain container 21 and the collection container 23 are both in the empty state. Further, it will be assumed that all the valves v1 to v5 start in the closed state in which they squash the corresponding pipes 2. Note that steps ST1 to ST4 included in the operation of the separation apparatus 1 each proceed under the control of the controller 30 without intervention by a user or the like.

In step ST1 which is a blood introducing step, the controller 30 controls the valves v1 and v4 to be in their respective opened states as shown in FIG. 5. The controller 30 thus forms a first flow channel fc11 through which the blood container bg and the filter 24 are in communication with each other, and also a drain flow channel fc12 through which the filter 24 and the drain container 21 are in communication with each other. The valve v1 is an example of a first valve for opening and closing the first flow channel fc11.

In this state, the controller 30 controls the piston pulling action of the drain container 21 so that the blood in the blood container bg is suctioned and moved toward the drain container 21 through the first flow channel fc11 and the drain flow channel fc12. The first flow channel fc11 thus introduces, in the anti-gravity direction, the blood from the blood container bg into the filter 24 through the inlet 24a located at the lower part of the filter 24. The filter 24 separates white blood cells contained in the blood introduced through the flow channel fc11 in the anti-gravity direction. The blood components that have passed through the filter 24, such as red blood cells and platelets, move upward in the anti-gravity direction and reach the drain container 21. At this time, the filter 24 is soaked with blood components, and the red blood cells as well as the trapped white blood cells remain at the filter 24. Air bubbles b2 in the filter 24 are lighter than the blood components, and thus, the air bubbles b2 move upward in the anti-gravity direction and exit from the outlet 24b. The discharged blood components and air bubbles b2 move through the drain flow channel fc12 and enter the drain container 21.

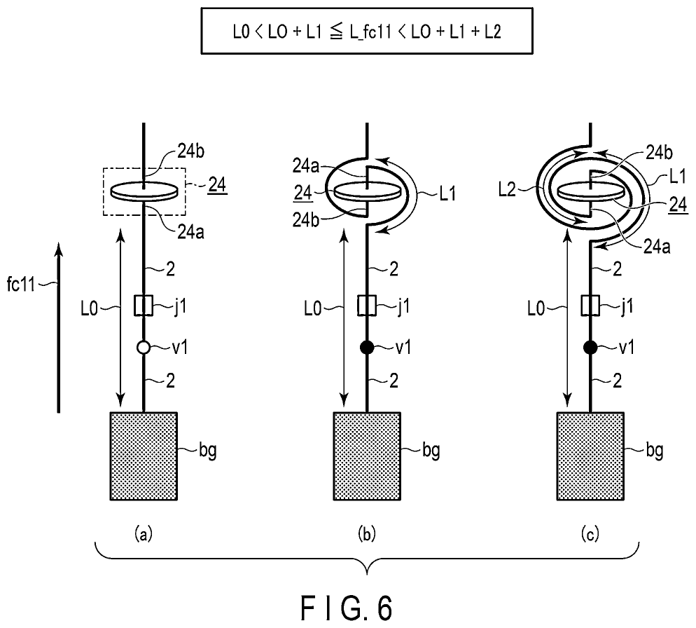

Note that, while the first flow channel fc11 is shown as a straight line in FIGS. 5 and 6, the first flow channel fc11 in implementation may have a length larger than the straight line to a given extent. In one example, the length of the first flow channel fc11, which is denoted by reference sign “L_fc11”, is larger than the shortest distance L0 between the blood container bg and the filter 24 as shown in FIG. 6(a) (L0<L_fc11). More specifically, the length L_fc11 of the first flow channel fc11 is equal to or larger than the length which enables, as shown in FIG. 6(b), the flipping over of the filter 24 once (L_fc11 >L0+L1). Also, the length L_fc11 of the first flow channel fc11 is smaller than the length which enables, as shown in FIG. 6(c), the flipping over of the filter 24 twice in the same direction (L_fc11<L0+L1+L2). Here, flipping over the filter 24 twice in the same direction means causing the filter 24 to make a full vertical rotation.

The controller 30, after the blood components resulting from the separation of white blood cells have flowed into the drain container 21, controls the valves v1 and v4 to be in their respective closed states so as to cancel the first flow channel fc11 and the drain flow channel fc12. Step ST1 is thus finished.

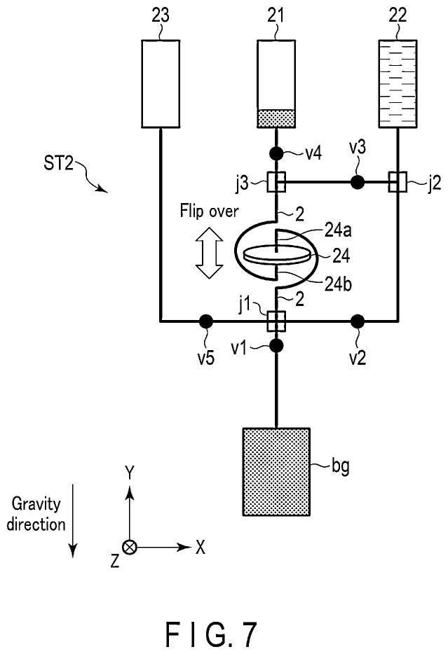

In step ST2 which is a flipping-over step, the controller 30 controls a flipping mechanism (not shown in the figure) to flip over the filter 24 that has undergone the introduction of the blood. This places the filter 24 in such an orientation that the inlet 24a is positioned at the upper part and the outlet 24b is positioned at the lower part as shown in FIG. 7. More specifically, in order to wash away the blood components soaked into the filter 24, the rinse liquid needs to be fed in the gravity direction since the red blood cells sink in the gravity direction. Thus, step ST2 first turns the filter 24 upside down. This flipping over of the filter 24 creates a state where the white blood cells have moved down in the gravity direction and settled at the material of the filter 24, while the red blood cells have moved down in the gravity direction and settled inside the filter container. In other words, step ST2 serves to form a state which enables easy removal of the red blood cells from the filter 24. Step ST2 is thus finished.

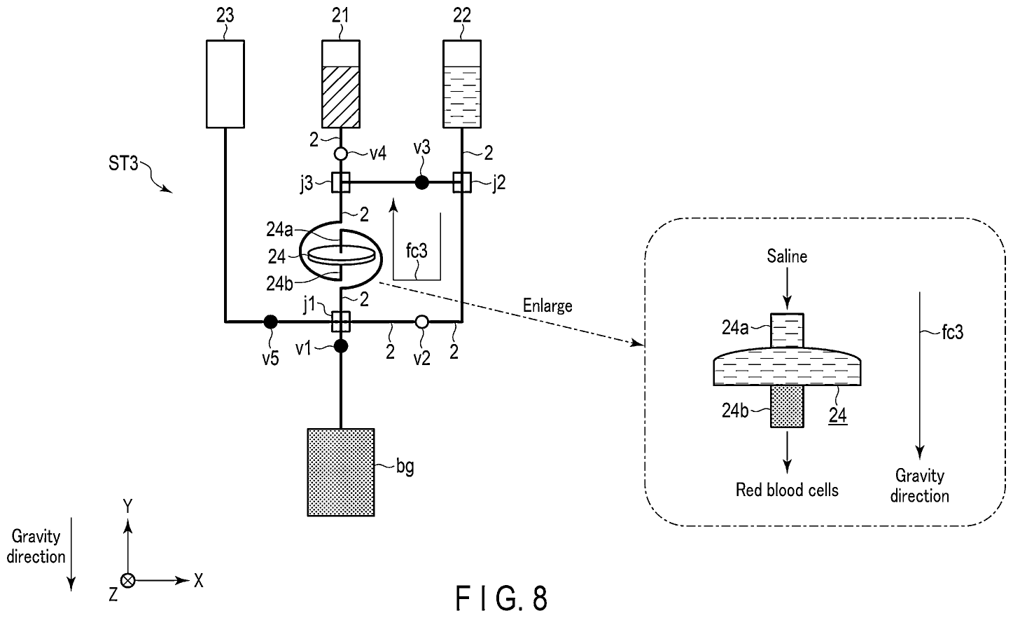

In step ST3 which is a rinsing step, the controller 30 controls the valves v2 and v4 to be in their respective opened states as shown in FIG. 8. The controller 30 thus forms a washing flow channel fc3 constituted by: a second flow channel through which the medical liquid container 22 and the joint j1 which is a part of the first flow channel fc11 are in communication with each other; a latter part of the first flow channel fc11 through which the joint j1 and the filter 24 are in communication with each other; and a drain channel through which the filter 24 and the drain container 21 are in communication with each other. The valve v2 is an example of a second valve for opening and closing the second flow channel.

In this state, the controller 30 controls the piston pushing action of the medical liquid container 22 and the piston pulling action of the drain container 21 in synchronization with each other. This causes the rinse liquid in the medical liquid container 22 to flow through the washing flow channel fc3 so as to be sent to the filter 24 via the joint j1. Here, the latter part of the first flow channel fc11 that is subsequent to the joint j1 introduces the rinse liquid coming from the second flow channel into the flipped-over filter 24 from above the filter 24 and in the gravity direction. The rinse liquid then passes through the filter 24 and is delivered to the drain container 21 through the washing flow channel fc3. Here, the red blood cells that have remained in the filter 24 are washed away in the gravity direction by the rinse liquid and move into the drain container 21. This places the filter 24 in a washed state while the filter 24 keeps holding the white blood cells. The flow channel between the joint j1 and the drain container 21 is also placed in a washed state.

Subsequently, the controller 30 controls the valves v2 and v4 to be in their closed states so as to cancel the washing flow channel fc3. Step ST3 is thus finished. Note that the controller 30 in this step may instead perform control such that only either one of the piston pulling action of the drain container 21 and the piston pushing action of the medical liquid container 22 is performed while the other piston action is forgone by leaving the corresponding piston unoperated without application of a force.

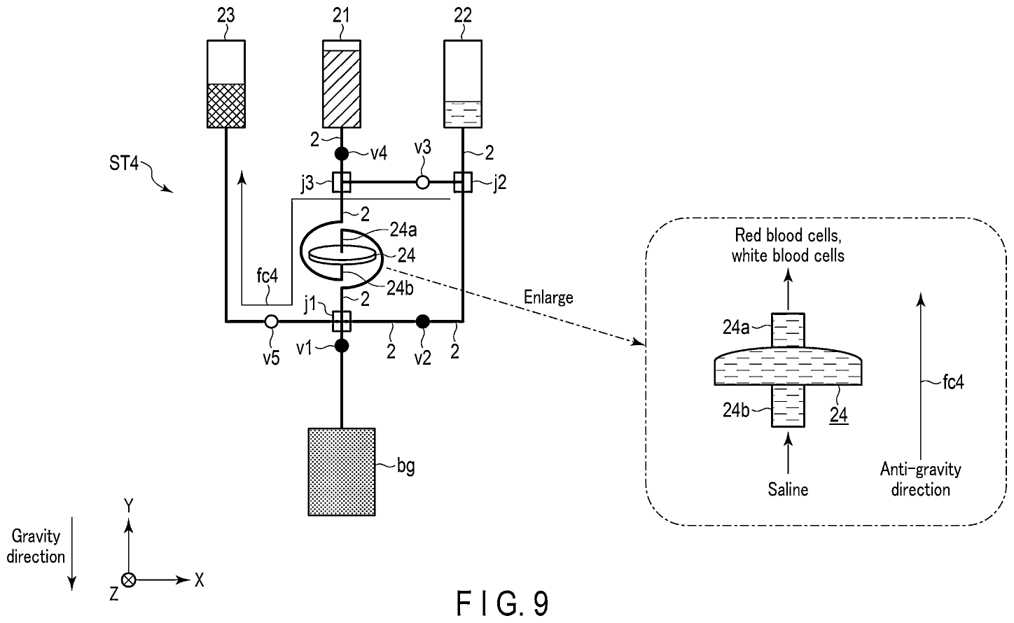

In step ST4 which is a collecting step, the controller 30 controls the valves v3 and v5 to be in their respective opened states as shown in FIG. 9. The controller 30 thus forms a collection flow channel fc4 constituted by: a third flow channel through which the medical liquid container 22 and the blood-discharging outlet 24b of the filter 24 are in communication with each other; and a fourth flow channel through which the blood-introducing inlet 24a of the filter 24 and the collection container 23 are in communication with each other.

In this state, the controller 30 controls the piston pulling action of the collection container 23 and the piston pushing action of the medical liquid container 22 in synchronization with each other. Accordingly, the third flow channel, i.e., the preceding part of the collection flow channel fc4, introduces the rinse liquid into the filter 24 that has once undergone the introduction of the rinse liquid, through the outlet 24b located at the lower part of the filter 24 and in the anti-gravity direction. The rinse liquid then passes through the filter 24 and is delivered to the collection container 23 together with the white blood cells through the collection flow channel fc4. That is, by causing the rinse liquid in the medical liquid container 22 to pass through the filter 24 from below and flow to the collection container 23, the white blood cells caught by the filter 24 are sent to and collected in the collection container 23 together with the rinse liquid.

Subsequently, the controller 30 controls the valves v3 and v5 to be in their closed states so as to cancel the collection flow channel fc4. Step ST4 is thus finished. Note that the controller 30 in this step may instead perform control such that only either one of the piston pulling action of the collection container 23 and the piston pushing action of the medical liquid container 22 is performed while the other piston action is forgone by leaving the corresponding piston unoperated without application of a force.

According to the first embodiment as described above, the separation apparatus 1 includes the filter 24 for separating white blood cells from blood sent from the blood container bg, and the first flow channel fc11 through which the blood container bg and the filter 24 are in communication with each other. The separation apparatus 1 introduces, through the first flow channel fc11, the blood into the filter 24 from below and in the anti-gravity direction. With this configuration of introducing blood into the filter 24 from below and in the anti-gravity direction, blood components passing through the filter 24 and air bubbles b2 present in the filter 24 are caused to move upward in the anti-gravity direction while the white blood cells are caught by the filter 24. Thus, the air bubbles b2 that would otherwise remain in the filter 24 in the course of introducing blood into the filter 24 can be eliminated or reduced.

Also according to the first embodiment, the separation apparatus 1 further includes the medical liquid container 22 for storing a rinse liquid (wash liquid) for washing the filter 24, the second flow channel through which the medical liquid container 22 and a part of the first flow channel fc11 are in communication with each other, and a flipping mechanism for flipping over the filter 24. The medical liquid container 22 is an example of a first medical liquid container for storing a wash liquid. The flipping mechanism flips over the filter 24 after the introduction of blood. The first flow channel fc11 that is in communication with the second flow channel introduces the rinse liquid coming from the second flow channel into the flipped-over filter 24 from above the filter 24 and in the gravity direction. Thus, the flipping over of the filter 24 enables the delivery of the rinse liquid in the gravity direction from above the filter 24, in the state where the white blood cells have moved down in the gravity direction and settled at the material of the filter 24 and the red blood cells have moved down in the gravity direction and settled inside the filter container. That is, it is possible to wash the filter 24 in the state where the red blood cells can be easily removed from the filter 24.

Also according to the first embodiment, the separation apparatus 1 includes the medical liquid container 22 serving as a second medical liquid container for storing a rinse liquid (collection liquid) for collecting white blood cells from the filter 24, and the third flow channel through which the medical liquid container 22 and the blood-discharging outlet 24b of the filter 24 are in communication with each other. The medical liquid container 22 is an example of the second medical liquid container for storing a collection liquid. The third flow channel introduces the rinse liquid (collection liquid) into the filter 24 that has undergone the introduction of the rinse liquid (wash liquid), through the outlet 24b located at the lower part of the filter 24 and in the anti-gravity direction. In this manner, white blood cells can be collected from the filter 24 by moving the white blood cells caught at the washed filter 24 in the anti-gravity direction together with the rinse liquid (collection liquid) and discharging them from the upper part of the filter 24.

Also according to the first embodiment, the wash liquid and the collection liquid are the same as each other, i.e., the rinse liquid (one liquid). The first medical liquid container and the second medical liquid container are the same as each other, i.e., the medical liquid container 22 (one container). Thus, in addition to the effects discussed above, the multi-purpose use of the liquid and the container can realize a space-saving apparatus at low cost.

Also according to the first embodiment, the filter 24 catches or traps white blood cells contained in blood, while permitting red blood cells and platelets contained in the blood to pass through. As such, in addition to the effects discussed above, separation of white blood cells from blood can include separation of the white blood cells from red blood cells and platelets.

Also according to the first embodiment, the first flow channel fc11 has a length larger than the shortest distance L0 between the blood container bg and the filter 24. Thus, in addition to the effects discussed above, the flipping over of the filter 24 is enabled with a simple configuration where the first flow channel fc11 has an extra length.

Also according to the first embodiment, the length L_fc11 of the first flow channel fc11 is smaller than the length (L0+L1+L2) which enables the flipping over of the filter 24 twice in the same direction. Thus, in addition to the effects discussed above, the flipping over of the filter 24 is enabled without making the length of the first flow channel fc11 unnecessarily long.

Also according to the first embodiment, the separation apparatus 1 includes the valve v1 for opening and closing the first flow channel fc11, the valve v2 for opening and closing the second flow channel, and the controller 30 for controlling the opening and closing actions of valves v1 and v2. The controller 30 controls, among the valves v1 and v2, only the valve v1 to be in its opened state so as to introduce blood into the filter 24. Also, the controller 30 controls, among the valves v1 and v2, only the valve v2 to be in its opened state so as to introduce the rinse liquid (wash liquid) into the filter 24. Thus, in addition to the effects discussed above, the configuration of the controller 30 performing the opening and closing control over the valves can realize automated valve opening and closing actions without an intervention by a user or the like.

Also according to the first embodiment, a detachable cartridge may be employed as the collector 20. Thus, in addition to the effects discussed above, this configuration allows the substantially-closed-system flow channels to be provided as a disposable product which can be discarded every time the white blood cells are collected from the blood of a donor. Accordingly, the cartridge can be exchanged for each donor blood, and a step for thoroughly washing the substantially-closed-system flow channels can be omitted, and therefore, the amount of collected white blood cells per unit time can be increased. That is, an improved throughput for the white blood cell collecting process can be realized.

Also according to the first embodiment, a cartridge serving as the collector 20 may include substantially-closed-system flow channels and a substantially-closed-system container which accommodates the flow channels. With this configuration, even in the event that the substantially-closed-system flow channels are damaged to cause the liquid to spill, the substantially-closed-system container does not allow such a spilled liquid to leak out. Thus, in addition to the effects discussed above, safety can be secured without necessitating a facility investment associated with the risk of liquid leakage. To be more specific, according to the first embodiment, it is possible to locally secure safety with the cartridge, rather than having to do so through a global facility such as a safety cabinet or a laboratory.

### Modification 1

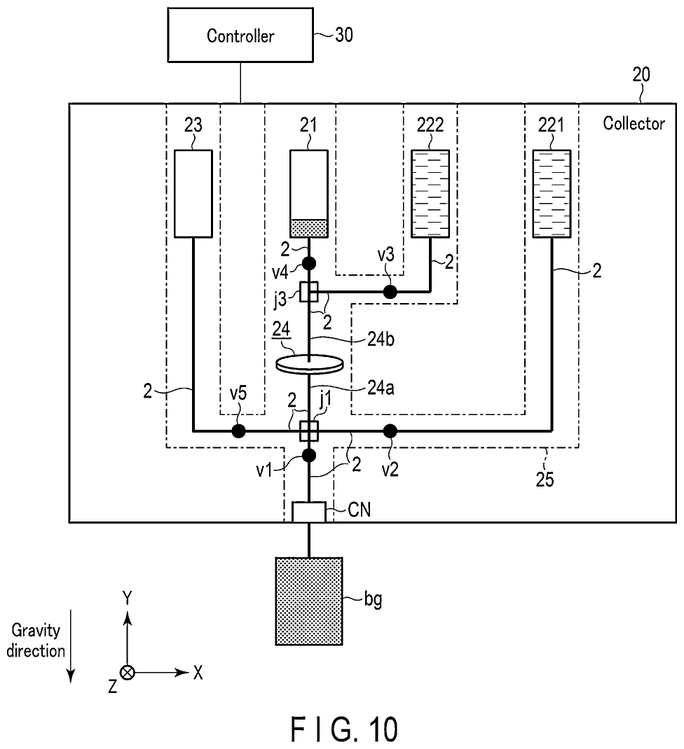

While the first embodiment has assumed that the wash liquid and the collection liquid are the same rinse liquid and that the first medical liquid container and the second medical liquid container are the same medical liquid container 22, no limitation is intended by this. For example, the wash liquid and the collection liquid may be discrete liquids differing from each other. Also, the first medical liquid container and the second medical liquid container may be discrete containers differing from each other. More specifically, and for example, the collector 20 may omit the joint j2 and include, in place of the medical liquid container 22, a first medical liquid container 221 for storing a rinse liquid, i.e., a wash liquid, and a second medical liquid container 222 for storing a collection liquid differing from the rinse liquid, as shown in FIG. 10.

In one example, the first medical liquid container 221 is similar to the medical liquid container 22 described above and is constituted by a syringe including a cylinder and a piston. The first medical liquid container 221 is adapted to contain a rinse liquid in its cylinder with an airtight configuration so that the rinse liquid is inhibited or prevented from being exposed to the ambient air. Also, the tip of the first medical liquid container 221 is connected to the joint j1 via the applicable pipe 2 which is opened and closed by the valve v2. The first medical liquid container 221 is controlled by the controller 30 to carry out a piston pushing action so as to discharge the rinse liquid present in the cylinder through its tip. The rinse liquid is, for example, a phosphate buffer saline solution (PBS) or the like, and a liquid which causes very little damage to white blood cells may be adopted as the rinse liquid. The first medical liquid container 221 as such is suitable for realizing quantitative and accurate liquid delivery. The rinse liquid is used as a wash liquid for washing the filter 24. The rinse liquid is an example of a liquid differing from the collection liquid.

In one example, the second medical liquid container 222 is constituted by a syringe including a cylinder and a piston. The second medical liquid container 222 is adapted to contain a collection liquid in its cylinder with an airtight configuration so that the collection liquid is inhibited or prevented from being exposed to the ambient air. Also, the tip of the second medical liquid container 222 is connected to the joint j3 via the applicable pipe 2 which is opened and closed by the valve v3. The second medical liquid container 222 is controlled by the controller 30 to carry out a piston pushing action so as to discharge the collection liquid present in the cylinder through its tip. The collection liquid is, for example, a culture medium and may be a liquid that is usable in the subsequent culturing step. The second medical liquid container 222 as such is suitable for realizing quantitative and accurate liquid delivery. The culture medium is an example of the collection liquid differing from the wash liquid.

The remaining configurations are the same as those in the first embodiment.

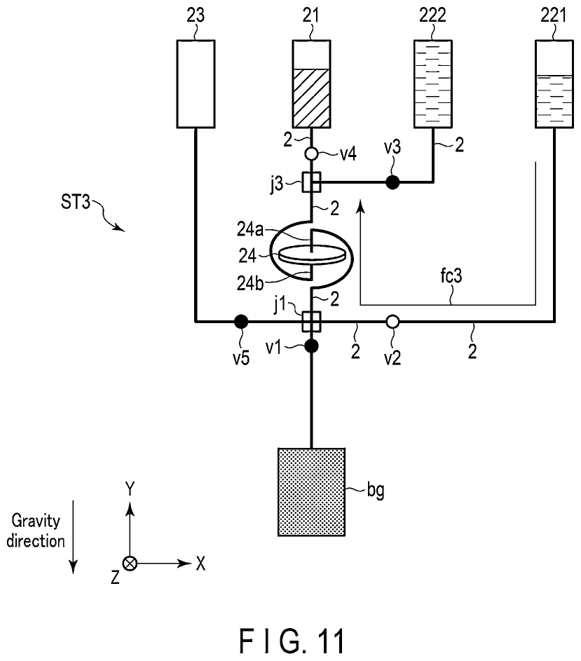

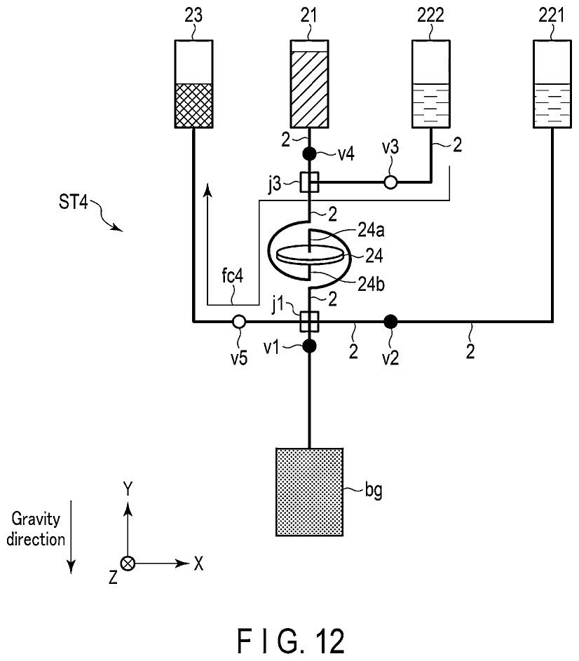

Next, an exemplary operation according to Modification 1 adopting the above configuration will be described with reference to the schematic diagrams in FIGS. 11 and 12. First, steps ST1 and ST2 are performed in a manner similar to the above.

In step ST3 which is a rinsing step after step ST2, the controller 30 controls the valves v2 and v4 to be in their opened states as shown in FIG. 11. The controller 30 thus forms: a second flow channel through which the first medical liquid container 221 and the joint j1 which is a part of the first flow channel fc11 are in communication with each other; a latter part of the first flow channel fc11 through which the joint j1 and the filter 24 are in communication with each other; and a drain channel through which the filter 24 and the drain container 21 are in communication with each other. The second flow channel, the latter part of the first flow channel fc11, and the drain channel together constitute a washing flow channel fc3. The valve v2 is an example of a second valve for opening and closing the second flow channel.

In this state, the controller 30 controls the piston pushing action of the first medical liquid container 221 and the piston pulling action of the drain container 21 in synchronization with each other. This causes the rinse liquid in the first medical liquid container 221 to flow through the washing flow channel fc3 so as to be sent to the filter 24 via the joint j1. Here, through the latter part of the first flow channel fc11 that is subsequent to the joint j1, the rinse liquid coming from the second flow channel is introduced into the flipped-over filter 24 from above the filter 24 and in the gravity direction. The rinse liquid then passes through the filter 24 and is delivered to the drain container 21 through the washing flow channel fc3. Accordingly, the filter 24 is washed while the caught white blood cells are kept as described above. Also, the flow channel between the joint j1 and the drain container 21 is likewise placed in a washed state.

Subsequently, the controller 30 controls the valves v2 and v4 to be in their closed states so as to cancel the washing flow channel fc3. Step ST3 is thus finished. Note that the controller 30 in this step may instead perform control such that only either one of the piston pulling action of the drain container 21 and the piston pushing action of the first medical liquid container 221 is performed while the other piston action is forgone by leaving the corresponding piston unoperated without application of a force.

In step ST4 which is a collecting step after step ST3, the controller 30 controls the valves v3 and v5 to be in their opened states as shown in FIG. 12. The controller 30 thus forms a collection flow channel fc4 constituted by: a third flow channel through which the second medical liquid container 222 and the blood-discharging outlet 24b of the filter 24 are in communication with each other; and a fourth flow channel through which the blood-introducing inlet 24a of the filter 24 and the collection container 23 are in communication with each other.

In this state, the controller 30 controls the piston pulling action of the collection container 23 and the piston pushing action of the second medical liquid container 222 in synchronization with each other. Accordingly, the third flow channel, i.e., the preceding part of the collection flow channel fc4, introduces the collection liquid into the filter 24 that has undergone the introduction of the rinse liquid, through the outlet 24b located at the lower part of the filter 24 and in the anti-gravity direction. The white blood cells caught by the filter 24 are therefore sent to the collection container 23 through the collection flow channel fc4 and collected there together with the collection liquid, in a manner similar to the above.

Subsequently, the controller 30 controls the valves v3 and v5 to be in their closed states so as to cancel the collection flow channel fc4. Step ST4 is thus finished. Note that the controller 30 in this step may instead perform control such that only either one of the piston pulling action of the collection container 23 and the piston pushing action of the second medical liquid container 222 is performed while the other piston action is forgone by leaving the corresponding piston unoperated without application of a force.

According to Modification 1 as described above, the wash liquid and the collection liquid are different from each other, and also the first medical liquid container and the second medical liquid container are different from each other. Thus, in addition to the effects discussed above, a liquid suitable for the later steps can be employed as the collection liquid, and a smooth proceeding of the later steps can therefore be expected. For example, if a culture medium for a subsequent culturing step is employed as the collection liquid, it can be expected that the subsequent culturing step will proceed smoothly.

### Modification 2

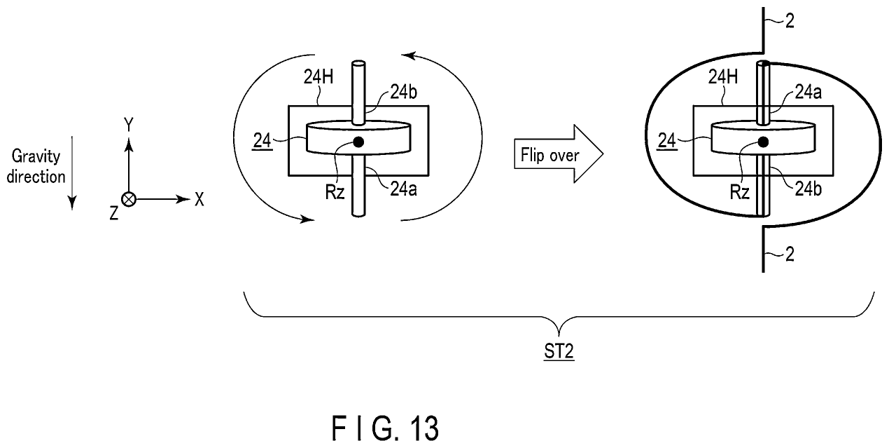

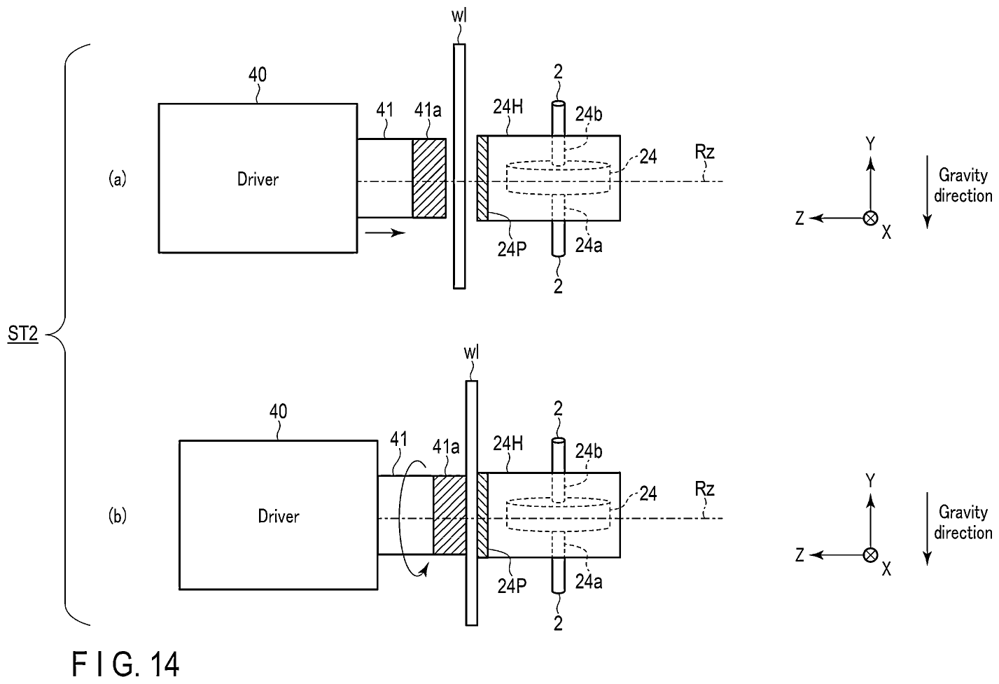

The first embodiment and its Modification 1 have been described without details of the flipping mechanism which flips over the filter 24. While no limitation is intended, an exemplary configuration of the flipping mechanism will be described. For example, as shown in FIG. 13, the flipping mechanism in step ST2 flips over the filter 24 by causing the filter 24 to make a half rotation about an axis Rz which is orthogonal to the straight line connecting the blood-introducing inlet 24a and the blood-discharging outlet 24b of the filter 24. More specifically, the collector 20 includes a holder 24H which holds the filter 24 as shown in FIGS. 13 and 14. Here, the filter 24, the holder 24H, the first flow channel fc11, the medical liquid container 22, and the second flow channel are accommodated in the cartridge, i.e., the collector 20. The holder 24H includes a holder body which is adapted to hold the filter 24 and made of a resin or plastic material, and to which a metal plate 24P is fixed in the vicinity of a part of a side wall wl of the cartridge. The metal plate 24P has a property of being attracted to a magnet. Note that the holder 24H is not limited to this and may adopt a structure where, for example, the holder body is made of a metal material.

On the other hand, the flipping mechanism includes a driver 40 for rotating a rotary shaft 41 which has a magnet 41a at its end. The magnet 41a may be covered by a protection cover (not shown in the figure) for protection. As shown in FIG. 14, the flipping mechanism flips over the filter 24 by pulling, through the cartridge side wall wl, the holder 24H with the metal plate 24P using the magnetic force of the magnet 41a, and then turning the pulled holder 24H upside down. More specifically, the flipping mechanism causes the rotary shaft 41 to make a half rotation about the axis Rz to accordingly cause the holder 24H to also make a half rotation following the half rotation of the magnet 41a fixed to the end of the rotary shaft 41 so that the flipping over of the filter 24 is conducted.

According to Modification 2 as described above, the flipping mechanism flips over the filter 24 by pulling the holder 24H through a part of the cartridge using magnetic force and then turning the pulled holder 24H upside down. Thus, in addition to the effects discussed above, the filter 24 inside the cartridge can be flipped over while the closed architecture of the cartridge is maintained.

### Second Embodiment

The second embodiment is a modification based on the first embodiment and replaces the respective syringes in the drain container 21 and the collection container 23 with liquid sending units each employing a reservoir connected to a pumping mechanism which produces a positive and/or negative gas pressure. Meanwhile, the medical liquid container 22 in this embodiment is assumed to have a syringe configuration as in the first embodiment, but the piston of the medical liquid container 22 is left unoperated without application of a force.

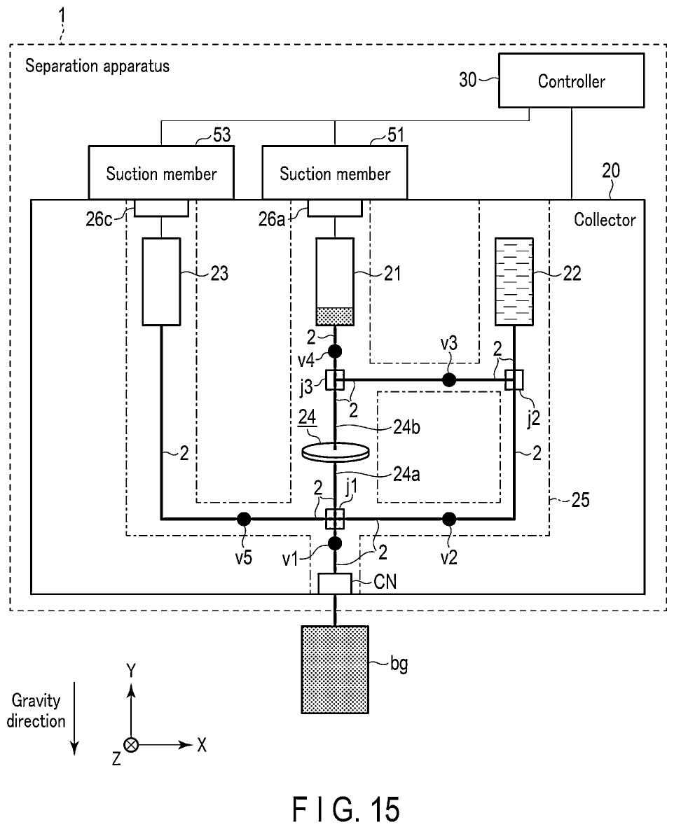

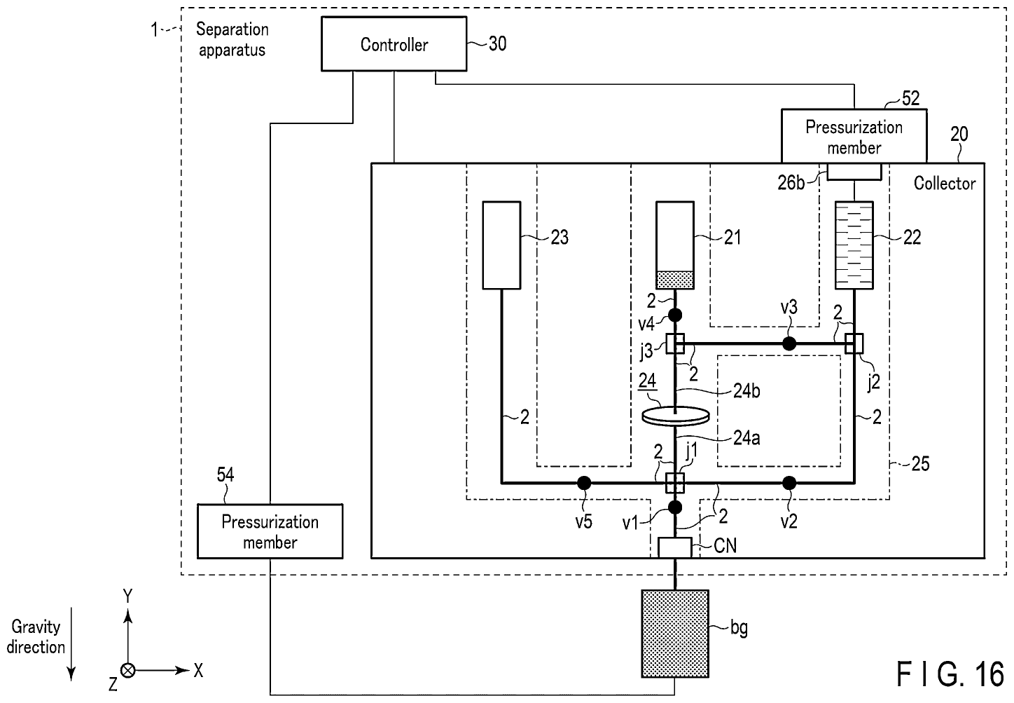

More specifically, as shown in FIG. 15, such liquid sending units may be constituted by the respective reservoirs of the drain container 21 and the collection container 23, and respective connectors 26a and 26c. The connectors 26a and 26c are in communication with the drain container 21 and the collection container 23, respectively, and are each in sterile communication with the outside of the collector 20 via an air filter. Here, as the pumping mechanisms, suction members 51 and 53 each adapted to produce a negative gas pressure may be employed. Each pumping mechanism may be constituted by a syringe pump or by a compressor and an air regulator, etc., and any suitable gas-producing mechanism may be employed as the pumping mechanism. The suction members 51 and 53 are coupled with the respective connectors 26a and 26c and perform a negative pressure applying action so that liquids are suctioned into the respective reservoirs of the drain container 21 and the collection container 23. More specifically, the suction member 51 applies a negative gas pressure to the drain container 21, so as to cause the blood in the blood container bg to be introduced into the filter 24 through the first flow channel fc11 and to suction the blood components that have passed through the filter 24 toward the drain container 21. The suction member 53 applies a negative gas pressure to the collection container 23, so as to cause the rinse liquid in the medical liquid container 22 to be introduced into the filter 24 and to suction the white blood cells that have been caught at the filter 24 toward the collection container 23.

In order to conduct steps ST1 to ST4 with the configuration of the second embodiment, the piston pulling actions of the drain container 21 and the collection container 23 discussed for the first embodiment may be replaced with the negative pressure applying actions to the respective reservoirs of the drain container 21 and the collection container 23. For example, the blood introducing step ST1 here includes controlling the suction member 51 to apply a negative pressure to the drain container 21, so as to cause the discharge of the blood from the blood container bg and to create a flow of the blood from the blood container bg toward the drain container 21. The rinsing step ST3 includes controlling the suction member 51 to apply a negative pressure to the drain container 21 while leaving the piston of the medical liquid container 22 unoperated, so as to create a flow of the rinse liquid from the medical liquid container 22 toward the drain container 21. The collecting step ST4 includes controlling the suction member 53 to apply a negative pressure to the collection container 23 while leaving the piston of the medical liquid container 22 unoperated, so as to create a flow of the rinse liquid from the medical liquid container 22 toward the collection container 23. While steps ST3 and ST4 here assume that the piston of the medical liquid container 22 is left unoperated, no limitation is intended by this. For example, in steps ST3 and ST4, the piston pushing action of the medical liquid container 22 may be performed in conjunction with the negative pressure applying actions to the drain container 21 and the collection container 23.

Also, while FIG. 15 shows a configuration where the suction members 51 and 53 are concurrently coupled with the respective connectors 26a and 26c, no limitation is intended by this. For example, the suction member 51 may be adapted to be movable so that the suction member 51 is moved and coupled with either of the connectors 26a and 26c that requires a gas pressure application in each of the steps ST1 to ST4.

According to the second embodiment as described above, the suction member 51 is provided to apply a negative gas pressure to the drain container 21 for storing the blood components that have passed through the filter 24. The suction member 51 applies a negative gas pressure to the drain container 21, so as to cause the blood in the blood container bg to be introduced into the filter 24 through the first flow channel fc11 and to suction the blood components that have passed through the filter 24 toward the drain container 21. Thus, in addition to the effects discussed above, a given moving mechanism, i.e., the syringe mechanism of the drain container 21, within the cartridge of the collector 20 can be omitted, and accordingly, more inexpensive manufacture of the cartridge of the collector 20 is enabled.

Similarly, according to the second embodiment, the suction member 53 is provided to apply a negative gas pressure to the collection container 23 for collecting the white blood cells that have been caught at the filter 24. The suction member 53 applies a negative gas pressure to the collection container 23, so as to cause the rinse liquid in the medical liquid container 22 to be introduced into the filter 24 and to suction the white blood cells picked up from the filter 24 and the rinse liquid toward the collection container 23. Thus, in addition to the effects discussed above, a given moving mechanism, i.e., the syringe mechanism of the collection container 23, within the cartridge of the collector 20 can be omitted, and accordingly, more inexpensive manufacture of the cartridge of the collector 20 is enabled.

### Modification 1

The second embodiment has assumed a configuration of applying a negative gas pressure to each of the drain container 21 and the collection container 23 to create flows of liquid directed toward the drain container 21 and the collection container 23, respectively, but no limitation is intended by this. For example, the blood container bg may be replaced with a liquid sending unit employing a reservoir connected to a pumping mechanism which produces a positive and/or negative gas pressure. Also, the syringe in the medical liquid container 22 may be replaced with a liquid sending unit employing a reservoir connected to a pumping mechanism which produces a positive and/or negative gas pressure. Meanwhile, the drain container 21 and the collection container 23 here are each assumed to have a syringe configuration as in the first embodiment, but each of the pistons of the drain container 21 and the collection container 23 is left unoperated without application of a force.

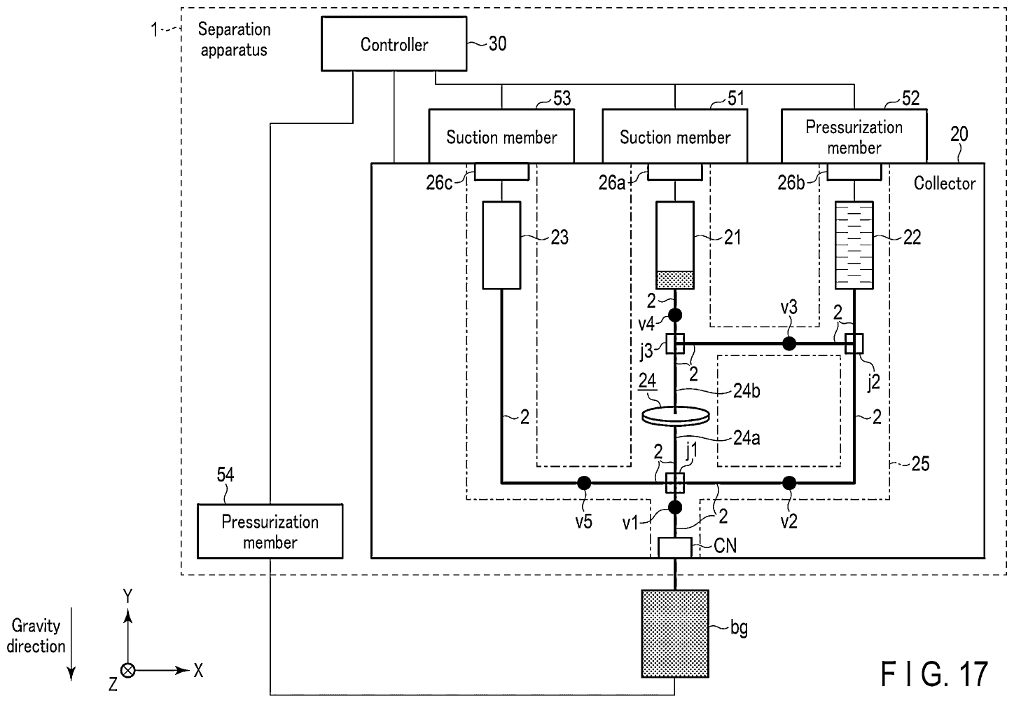

More specifically, as shown in FIG. 16, the liquid sending unit that replaces the syringe of the medical liquid container 22 may be constituted by the reservoir of the medical liquid container 22, and a connector 26b which is in communication with the medical liquid container 22 and also in sterile communication with the outside of the collector 20 via an air filter. Here, as the pumping mechanism, a pressurization member 52 adapted to produce a positive gas pressure may be employed. Also, as the pumping mechanism connected to the blood container bg, a pressurization member 54 adapted to produce a positive gas pressure may be employed. Each pumping mechanism may be constituted by a syringe pump or by a compressor and an air regulator, etc., and any suitable gas-producing mechanism may be employed as the pumping mechanism. The pressurization members 52 and 54 perform a positive pressure applying action so that liquids are discharged from the respective reservoirs of the blood container bg and the medical liquid container 22. More specifically, the pressurization member 54 applies a positive gas pressure to the blood container bg, so as to cause the blood in the blood container bg to be introduced into the filter 24 through the first flow channel fc11. The pressurization member 52 applies a positive gas pressure to the medical liquid container 22, so as to cause the rinse liquid in the medical liquid container 22 to be introduced into the filter 24.

In order to conduct steps ST1 to ST4 with the configuration of this Modification 1, the piston pulling actions of the drain container 21 and the collection container 23 discussed for the first embodiment may be replaced with the positive pressure applying actions to the respective reservoirs of the blood container bg and the medical liquid container 22. For example, the blood introducing step ST1 here includes controlling the pressurization member 54 to apply a positive pressure to the blood container bg, so as to cause the discharge of the blood from the blood container bg and to create a flow of the blood from the blood container bg toward the drain container 21. The rinsing step ST3 includes controlling the pressurization member 52 to apply a positive pressure to the medical liquid container 22 while leaving the piston of the drain container 21 unoperated, so as to cause the discharge of the rinse liquid from the medical liquid container 22 and to create a flow of the rinse liquid from the medical liquid container 22 toward the drain container 21. The collecting step ST4 includes controlling the pressurization member 52 to apply a positive pressure to the medical liquid container 22 while leaving the piston of the collection container 23 unoperated, so as to cause the discharge of the rinse liquid from the medical liquid container 22 and to create a flow of the rinse liquid from the medical liquid container 22 toward the collection container 23. While steps ST3 and ST4 here assume that the pistons of the drain container 21 and the collection container 23 are left unoperated, no limitation is intended by this. For example, in steps ST3 and ST4, the piston pulling actions of the drain container 21 and the collection container 23 may be performed in conjunction with the positive pressure applying action to the medical liquid container 22.

According to Modification 1 as described above, the pressurization member 54 is provided to apply a positive gas pressure to the blood container bg. The pressurization member 54 applies a positive gas pressure to the blood container bg, so as to cause the blood in the blood container bg to be introduced into the filter 24 through the first flow channel fc11. Similarly, according to Modification 1, the pressurization member 52 is provided to apply a positive gas pressure to the medical liquid container 22. The pressurization member 52 applies a positive gas pressure to the medical liquid container 22, so as to cause the rinse liquid in the medical liquid container 22 to be introduced into the filter 24 and to direct the liquid that has passed through the filter 24 toward the drain container 21 or the collection container 23. Thus, in addition to the effects discussed above, a given moving mechanism, i.e., the syringe mechanism of the medical liquid container 22, within the cartridge of the collector 20 can be omitted, and accordingly, more inexpensive manufacture of the cartridge of the collector 20 is enabled.

### Modification 2

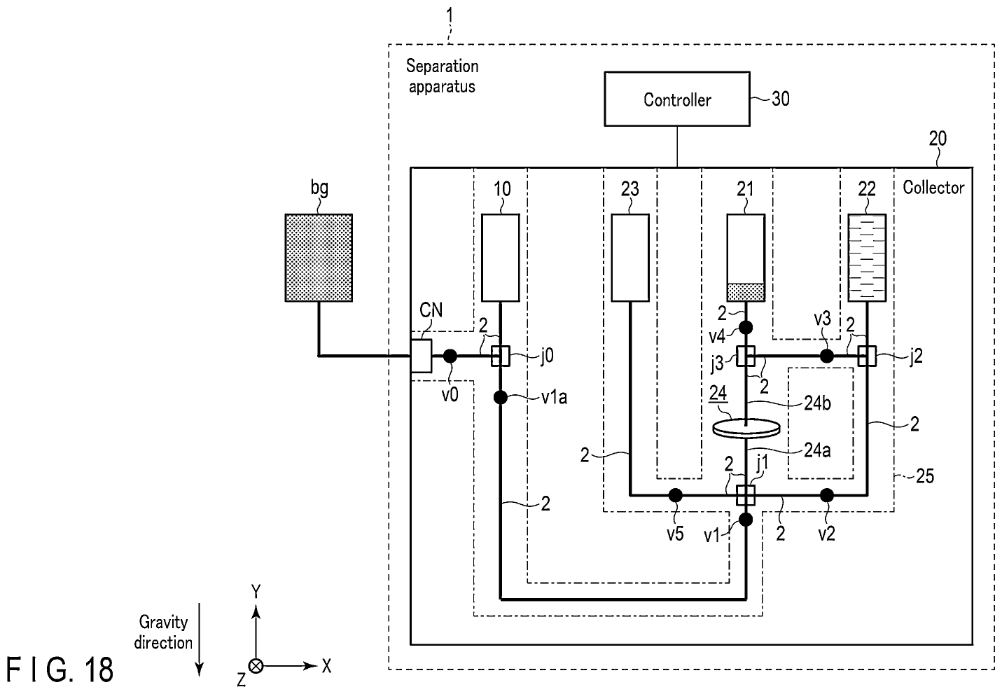

The second embodiment and its Modification 1 have assumed configurations that employ one of the set of the suction members 51 and 53 and the connectors 26a and 26c and the set of the pressurization members 52 and 54 and the connector 26b, but no limitation is intended by this. For example, the separation apparatus 1 may include, as shown in FIG. 17, both of the set of the suction members 51 and 53 and the connectors 26a and 26c and the set of the pressurization members 52 and 54 and the connector 26b. According to such Modification 2, it is possible to obtain the effects and advantages of both of the second embodiment and its Modification 1.

### Third Embodiment

The third embodiment is a modification based on the first embodiment and/or the second embodiment, and it replaces the configuration of introducing blood from the externally provided blood container bg into the filter 24 with a configuration of first suctioning the blood from the externally provided blood container bg to an intermediate container and then introducing the blood from this intermediate container into the filter 24.

More specifically, and for example, the collector 20 here differs from the configuration shown in FIG. 1 in that it further includes, as shown in FIG. 18, an intermediate container 10 arranged between the blood container bg and the filter 24 and adapted to temporarily store the blood from the blood container bg and send out the blood toward the filter 24. The intermediate container 10 is, accordingly, in communication with the above described joint j1 via the applicable pipe 2, a joint j0, and another applicable pipe 2. The joint j0 has a substantially T shape with branching portions, and is connected to three pipes 2 so that these three pipes 2 are in communication with one another. More specifically, the joint j0 places three pipes 2 which are respectively connected to the intermediate container 10, the sterile connector CN, and the joint j1, in a mutually communicating state. The pipe 2 between the joints j0 and j1 is opened and closed by synchronizing valves v1a and v1. The valve v1a is disposed near the joint j0 and the valve v1 is disposed near the joint j1. The pipe 2 between the joint j0 and the sterile connector CN is opened and closed by the valve v0. The sterile connector CN is freely attachable to and detachable from the pipe 2 for the blood container bg as in the foregoing embodiment, etc.

The intermediate container 10 in one example is constituted by a syringe including a cylinder and a piston. The intermediate container 10 is adapted to contain a liquid such as blood in its cylinder with an airtight configuration so that the liquid is inhibited or prevented from being exposed to the ambient air. The tip of the intermediate container 10 is connected to the joint j0 via the applicable pipe 2. The intermediate container 10 is controlled by the controller 30 to carry out a piston pulling action so as to take in the blood present in the blood container bg through its tip and a piston pushing action so as to discharge the blood present in the cylinder through the tip. The intermediate container 10 as such is suitable for realizing quantitative and accurate liquid delivery, and is used in a similar manner to the blood container bg described above in the state of storing blood. The intermediate container 10 is another example of a blood container.

The remaining configurations are the same as those in the first embodiment.

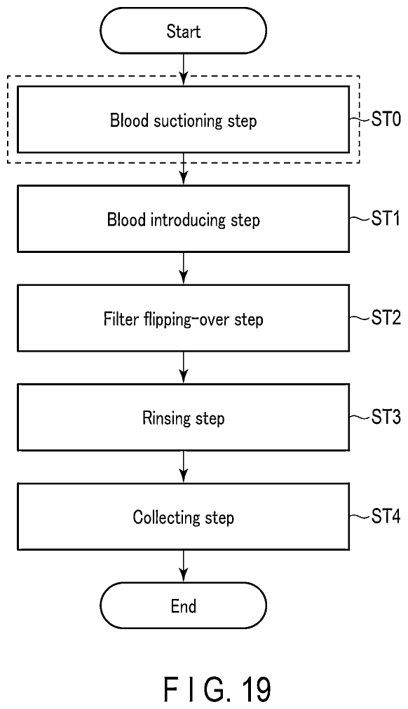

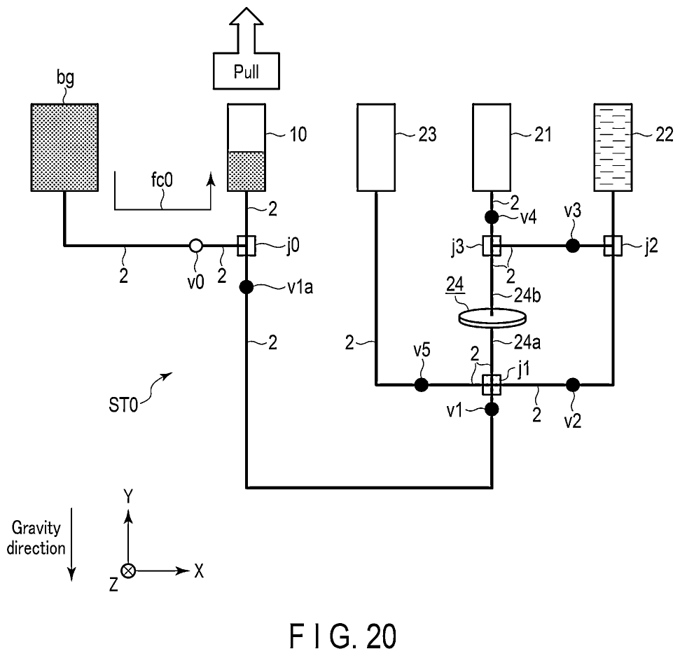

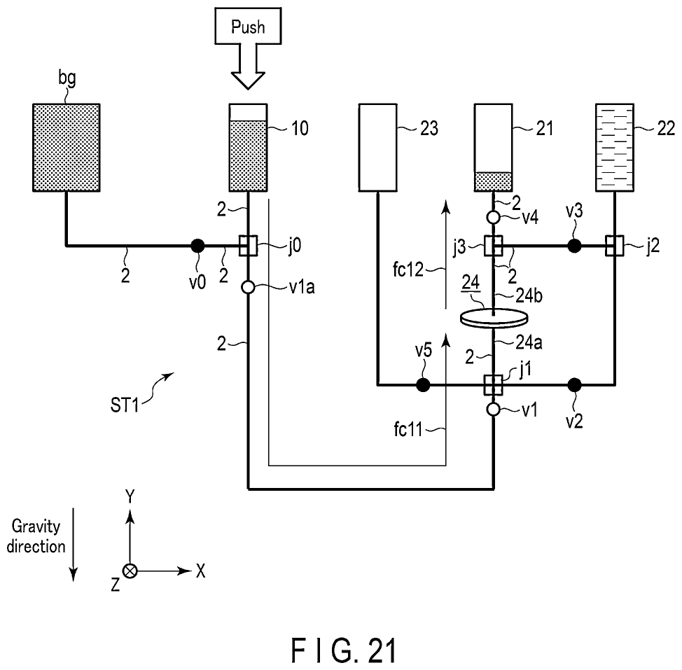

Next, an exemplary operation of the separation apparatus 1 configured as above will be described with reference to the flowchart in FIG. 19 and the schematic diagrams in FIGS. 20 and 21. As in the foregoing examples, it will be assumed that all the valves v0 to v5 start in the closed state in which they squash the corresponding pipes 2. Note also that steps ST0 to ST4 included in the operation of the separation apparatus 1 each proceed under the control of the controller 30 without intervention by a user or the like.

In step ST0 which is a blood suctioning step, the controller 30 controls the valve v0 to be in its opened state so as to form a suction flow channel fc0 from the blood container bg to the intermediate container 10 via the applicable pipe 2, the valve v0, the joint j0, and another applicable pipe 2 as shown in FIG. 20. Also, the controller 30 controls the piston pulling action of the intermediate container 10 so that the blood in the blood container bg is suctioned and moved to the intermediate container 10 through the suction flow channel fc0. The controller 30 thus transfers a given amount of the blood from the blood container bg to the intermediate container 10. Then, the controller 30 controls the valve v0 to be in its closed state so as to cancel the suction flow channel fc0. Step ST0 is thus finished. In subsequent step ST1, the intermediate container 10 functions in a manner similar to the blood container bg in step ST1 of the first embodiment.

That is, in the blood introducing step ST1, the controller 30 controls the valves v1a, v1, and v4 to be in their opened states as shown in FIG. 21. The controller 30 thus forms a first flow channel fc11 through which the intermediate container 10 and the filter 24 are in communication with each other, and also a drain flow channel fc12 through which the filter 24 and the drain container 21 are in communication with each other.

In this state, the controller 30 controls the piston pushing action of the intermediate container 10 and the piston pulling action of the drain container 21 in synchronization with each other. This causes the blood in the intermediate container 10 to flow through the first flow channel fc11 and the drain flow channel fc12 toward the drain container 21. Here, the first flow channel fc11 introduces, in the anti-gravity direction, the blood from the intermediate container 10 into the filter 24 through the inlet 24a located at the lower part of the filter 24. The filter 24 separates white blood cells contained in the introduced blood. The blood components that have passed through the filter 24, such as red blood cells and platelets, move upward in the anti-gravity direction and reach the drain container 21. The filter 24 here provides the same effects and advantages as described for the first embodiment.

The controller 30, after the blood components resulting from the separation of white blood cells have been sent to the drain container 21, controls the valves v1a, v1, and v4 to be in their respective closed states so as to cancel the first flow channel fc11 and the drain flow channel fc12. Step ST1 is thus finished. Note that the controller 30 in this step may instead perform control such that only either one of the piston pulling action of the drain container 21 and the piston pushing action of the intermediate container 10 is performed while the other piston action is forgone by leaving the corresponding piston unoperated without application of a force.

Subsequently, step ST2 and onward are performed in the same manner as described above.

According to the third embodiment as described above, the separation apparatus 1 further includes the intermediate container 10 arranged between the blood container bg and the filter 24 and adapted to temporarily store the blood from the blood container bg and send out the blood toward the filter 24. Accordingly, in addition to the effects discussed above, the configuration of temporarily storing the blood from the blood container bg and sending out the blood toward the filter 24 can realize quantitative and accurate liquid delivery by the syringe of the intermediate container 10.

### Modification 1

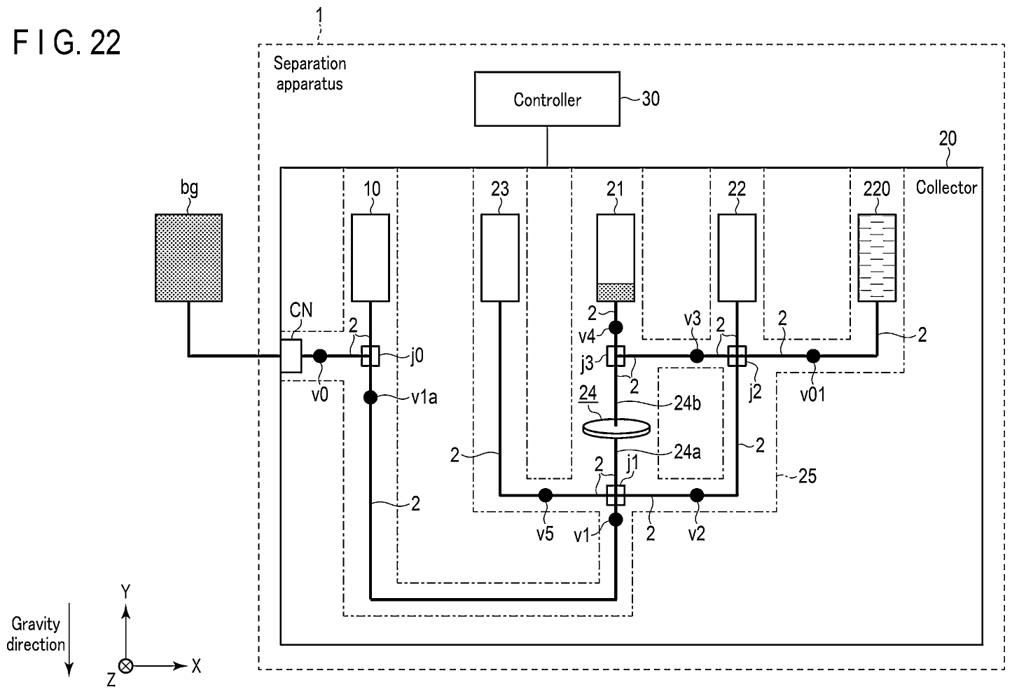

While the third embodiment has assumed inclusion of the intermediate container 10 for the blood container bg, no limitation is intended by this. For example, as shown in FIG. 22, the collector 20 may use the medical liquid container 22 as an intermediate container, while further including a third medical liquid container 220 for supplying the rinse liquid to the medical liquid container 22. That is, the medical liquid container 22 serving as an intermediate container here may suction the rinse liquid from the third medical liquid container 220, store the rinse liquid, and send out the rinse liquid toward the filter 24.

Accordingly, the third medical liquid container 220 is in communication with the joint j2 via the pipe 2 that has a valve v01. The joint j2 has a substantially crisscross shape with branching portions, and is connected to four pipes 2 so that these four pipes 2 are in communication with one another. More specifically, the joint j2 places four pipes 2 which are respectively connected to the third medical liquid container 220, the medical liquid container 22, the joint j1 and the joint j3, in a mutually communicating state. The pipe 2 between the third medical liquid container 220 and the joint j2 is opened and closed by the valve v01.

In one example, the third medical liquid container 220 is constituted by a syringe including a cylinder and a piston. The third medical liquid container 220 is adapted to contain a liquid such as a rinse liquid in its cylinder with an airtight configuration so that the liquid is inhibited or prevented from being exposed to the ambient air. The tip of the third medical liquid container 220 is connected to the joint j2 via the applicable pipe 2. The third medical liquid container 220 is controlled by the controller 30 to carry out a piston pushing action so as to discharge the rinse liquid present in the cylinder through its tip. The third medical liquid container 220 as such is suitable for realizing quantitative and accurate liquid delivery.

The remaining configurations are the same as those in the third embodiment.

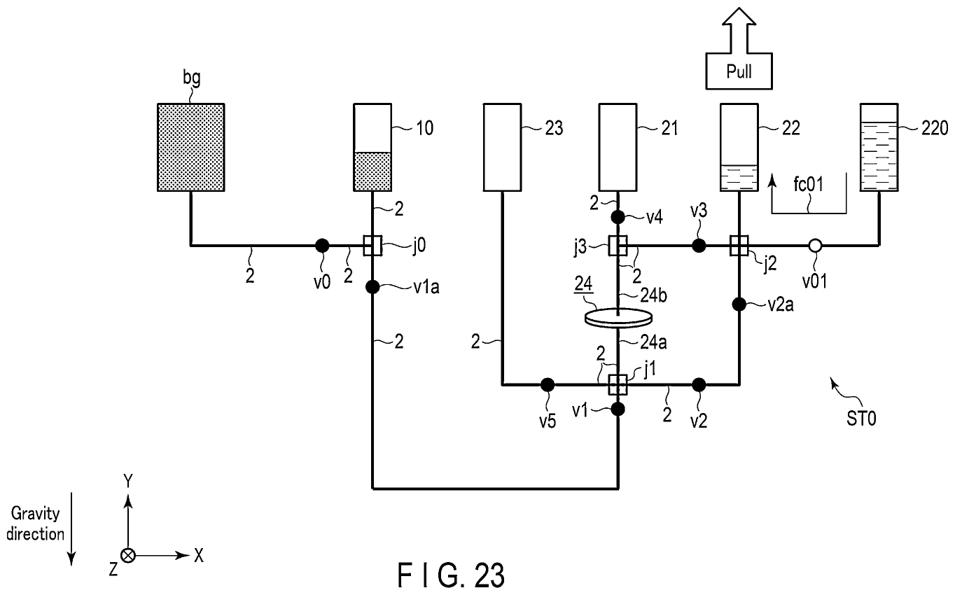

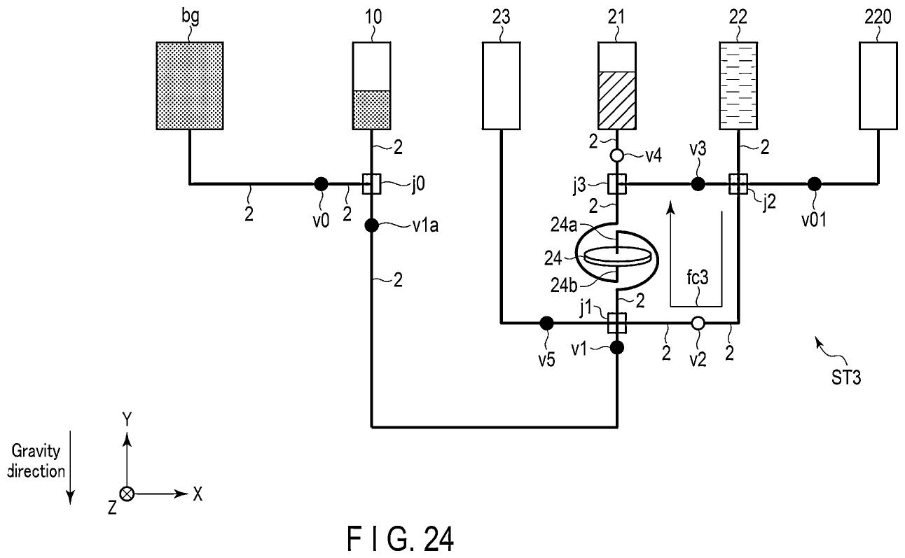

Next, an exemplary operation of the separation apparatus 1 configured as above will be described with reference to the schematic diagrams in FIGS. 23 and 24. As in the foregoing examples, it will be assumed that all the valves v01 and v0 to v5 start in the closed state in which they squash the corresponding pipes 2. Note also that steps ST0 to ST4 included in the operation of the separation apparatus 1 each proceed under the control of the controller 30 without intervention by a user or the like.

In the blood suctioning step ST0, the controller 30 transfers a given amount of blood from the blood container bg to the intermediate container 10 as described above with reference to FIG. 20. Then, the controller 30 controls the valve v0 to be in its closed state so as to cancel the suction flow channel fc0. Subsequently, the controller 30 controls the valve v01 to be in its opened state so as to form a suction flow channel fc01 from the third medical liquid container 220 to the medical liquid container 22 via the applicable pipe 2, the valve v01, the joint j2, and another applicable pipe 2 as shown in FIG. 23. Also, the controller 30 controls the piston pushing action of the third medical liquid container 220 and the piston pulling action of the medical liquid container 22 in synchronization with each other. This causes a given amount of the rinse liquid in the third medical liquid container 220 to flow through the suction flow channel fc01 so as to be sent to the medical liquid container 22. Then, the controller 30 controls the valve v01 to be in its closed state so as to cancel the suction flow channel fc01. Step ST0 is thus finished. Note that the controller 30 in this step may instead perform control such that only either one of the piston pulling action of the medical liquid container 22 and the piston pushing action of the third medical liquid container 220 is performed while the other piston action is forgone by leaving the corresponding piston unoperated without application of a force.

Subsequently, step ST1 and onward are performed in the same manner as described for the third embodiment. For example, the rinsing step ST3 which uses the medical liquid container 22 as an intermediate container proceeds as shown in FIG. 24, i.e., in a manner similar to the step shown in FIG. 8. Also for example, the collecting step ST4 which uses the medical liquid container 22 as an intermediate container proceeds in a manner similar to the step shown in FIG. 9.

According to Modification 1 as described above, the separation apparatus 1 uses the medical liquid container 22 as an intermediate container and further includes a third medical liquid container 220 for supplying the rinse liquid to the medical liquid container 22. Such a configuration of using the medical liquid container as an intermediate container can also provide the same effects as described above.

### Modification 2

Modification 1 has assumed inclusion of the third medical liquid container 220 in the collector 20, but this does not pose any limitation. As one example, for the separation apparatus 1, the third medical liquid container 220 may be provided as an external member, and the collector 20 may include a sterile connector CN for freely attaching or detaching the third medical liquid container 220. According to such Modification 2, the collector 20, which is a cartridge, does not store a liquid within it before the start of step ST0 since the drain container 21, the medical liquid container 22, and the collection container 23 are all empty then, and therefore, the collector 20 is light-weighted and suited for long-term storage. Moreover, the collector 20 in step ST0 is attached to the externally provided blood container bg and the externally provided third medical liquid container 220 via the respective sterile connectors CN, and the blood and the rinse liquid are accordingly suctioned to the respective intermediate containers. Thus, such Modification 2 allows for the use of fresh blood and rinse liquid for each step.

### Fourth Embodiment

The fourth embodiment is a modification based on one or more of the first to third embodiments, and it adopts a configuration of extracting CD34 positive cells from the white blood cells which have been collected from the filter 24 to the collection container 23 and then delivered from the collection container 23.

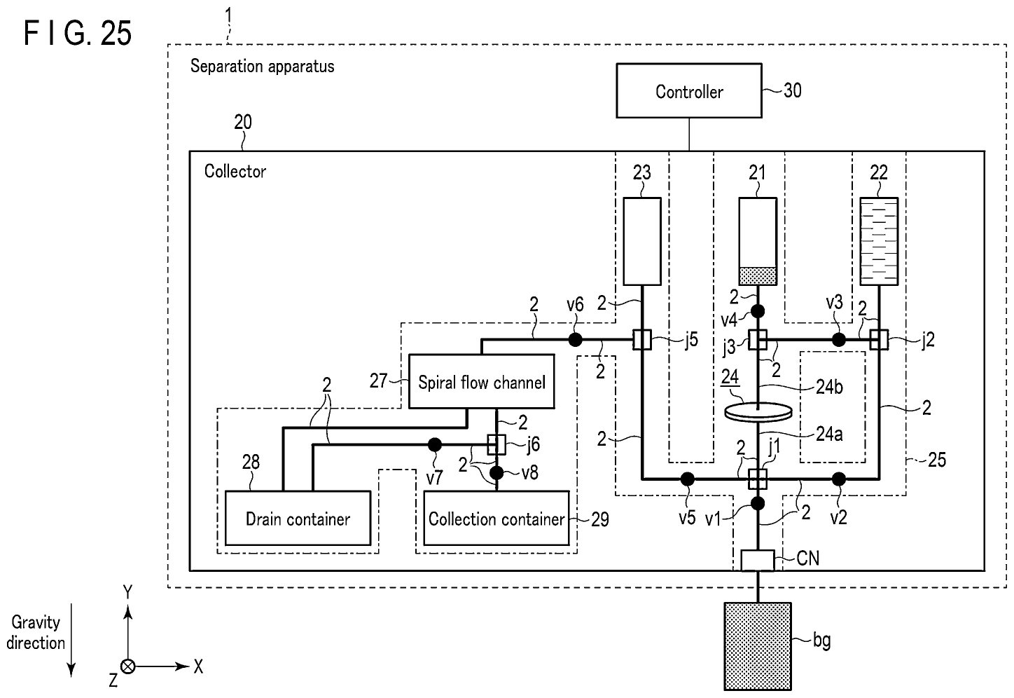

For example, the collector 20 here includes, as shown in FIG. 25, a spiral flow channel 27 for extracting CD34 positive cells from the white blood cells sent from the collection container 23, a drain container 28 for storing a drained liquid remaining after the extraction, and a collection contained for storing the extracted CDC34 positive cells. The spiral flow channel 27 is, for the introduction of the white blood cells, in communication with a joint j5 via the pipe 2 that has a valve v6. The spiral flow channel 27 is also in communication with the drain container 28 via another pipe 2, for the discharge of the drained liquid. Further, for the collection of the CD34 positive cells, the spiral flow channel 27 is in communication with the drain container 28 via yet another pipe 2, a joint j6, and the pipe 2 that has a valve v7, and in communication with the collection container 29 via the joint j6 and the pipe 2 that has a valve v8. The joints j5 and j6 each have a substantially T shape with branching portions, and they are each connected to three pipes 2 so that these three pipes 2 are in communication with one another. More specifically, the joint j5 places three pipes 2 which are respectively connected to the joint j1, the collection container 23, and the spiral flow channel 27, in a mutually communicating state. The joint j6 places three pipes 2 which are respectively connected to the spiral flow channel 27, the drain container 28, and the collection container 29, in a mutually communicating state.

Here, the spiral flow channel 27 is a spirally or helically shaped flow channel adapted to receive the liquid containing white blood cells and separate, according to the size of the particles contained in the liquid, the particles from the liquid. More specifically, the spiral flow channel 27 is adapted to extract CD34 positive cells from white blood cells delivered from the collection container 23 and flowing through the spirally or helically shaped flow channel, and send out the extracted CD34 positive cells to the collection container 29 and the remaining white blood cells, i.e., a drained liquid, to the drain container 28. The spiral flow channel 27 may be called a “spiral inertial filtering device”, or a “liquid processing device”. Note that the techniques of a spiral inertial filtering device are available from, for example, the disclosures in Jpn. PCT National Publication No. 2015-532198.

The drain container 28 is a container for storing a drained liquid. The drain container 28 receives the drained liquid from the spiral flow channel 27. The drain container 28 may be a flask, a bag, or any container that can receive and keep the drained liquid.

The collection container 29 is a container for storing CD34 positive cells included in the collected white blood cells. The collection container 29 receives the CD34 positive cells extracted by the spiral flow channel 27. The collection container 29 may be a tapered container, a well plate, a flask, a dish, a bag, or any container that can be used to culture CD34 positive cells.

The remaining configurations are the same as those in the first embodiment, etc.

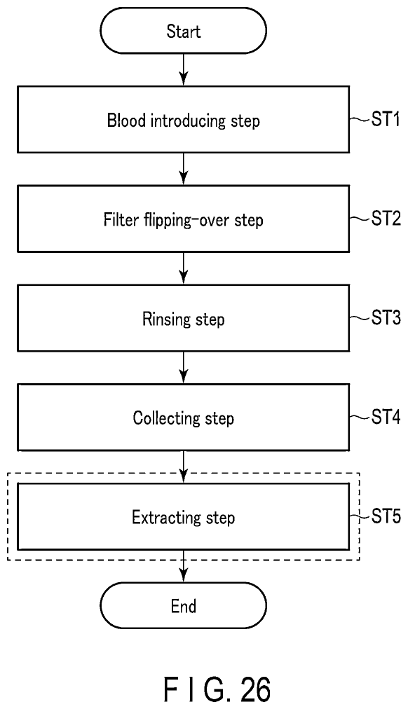

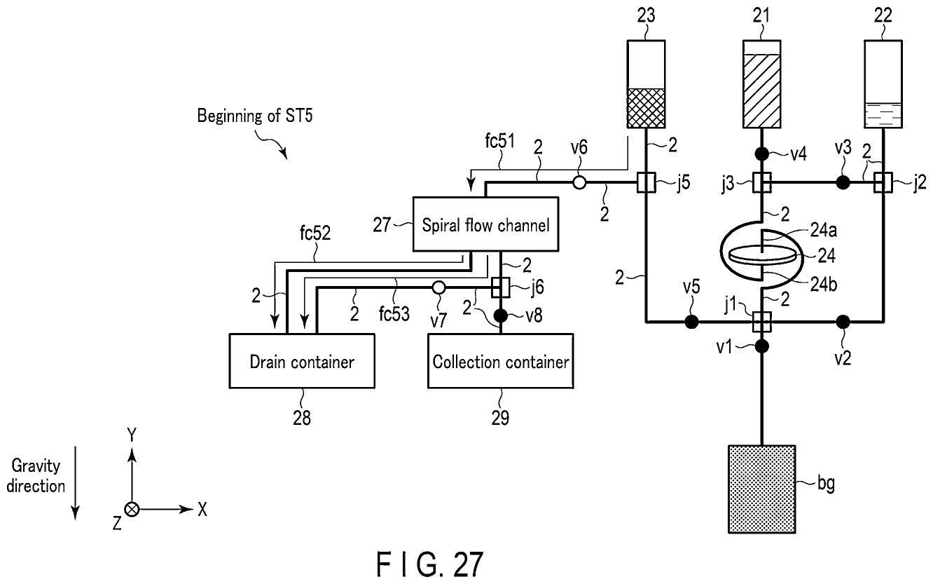

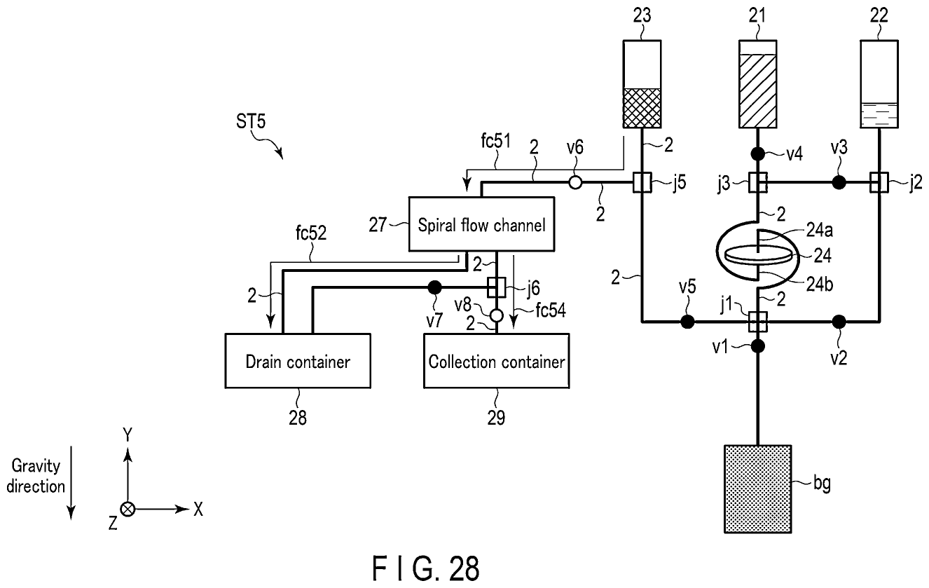

Next, an exemplary operation of the separation apparatus 1 configured as above will be described with reference to the flowchart in FIG. 26 and the schematic diagrams in FIGS. 27 and 28. As in the foregoing examples, it will be assumed that all the valves v1 to v7 start in the closed state in which they squash the corresponding pipes 2. Note that steps ST1 to ST5 included in the operation of the separation apparatus 1 each proceed under the control of the controller 30 without intervention by a user or the like.

Steps ST1 to ST4 are performed in a manner similar to the above. Finishing step ST4 places the collection container 23 in a state of storing the rinse liquid containing the white blood cells collected from the filter 24.

In step ST5 which is an extracting step, the controller 30 controls the valves v6 and v7 to be in their respective opened states as shown in FIG. 27. The opened state of the valve v6 forms a 51st flow channel fc51 from the collection container 23 to the spiral flow channel 27 via the applicable pipe 2, the joint j5, and another applicable pipe 2. There is a 52nd flow channel fc52 which is present all the time and extends from the spiral flow channel 27 to the drain container 28 via the applicable pipe 2. Also, the opened state of the valve v7 forms a 53rd flow channel fc53 from the spiral flow channel 27 to the drain container 28 via the applicable pipe 2, the joint j6, and another applicable pipe 2. In this state, the controller 30 controls the piston pushing action of the collection container 23. The controller 30 thus sends the rinse liquid containing the white blood cells from the collection container 23 to the spiral flow channel 27 through the 51st flow channel fc51, and then sends the rinse liquid that has passed through the spiral flow channel 27 to the drain container 28 individually through the 52nd flow channel fc52 or the 53rd flow channel fc53. Note that FIG. 27 illustrates an earlier operation in step ST5. In the beginning of step ST5, the rinse liquid passing through the spiral flow channel 27 has a low flow rate, and as such, CD34 positive cells are not extracted from the white blood cells contained in the rinse liquid and the rinse liquid is therefore sent out to the drain container 28 through the 53rd flow channel fc53.

Next, the controller 30 controls the valve v7 to be in the closed state and the valve v8 to be in the opened state as shown in FIG. 28. The closed state of the valve v7 cancels the 53rd flow channel fc53. On the other hand, the opened state of the valve v8 forms a 54th flow channel fc54 from the spiral flow channel 27 to the collection container 29 via the applicable pipe 2, the joint j6, and another applicable pipe 2. In this state, the controller 30 controls the continuous piston pushing action of the collection container 23. This causes the rinse liquid containing the white blood cells in the collection container 23 to flow through the 51st flow channel fc51 so as to be sent to the spiral flow channel 27. The rinse liquid that has passed through the spiral flow channel 27 is then sent to the drain container 28 through the 52nd flow channel fc52. Meanwhile, the CD34 positive cells extracted by the spiral flow channel 27 are sent to the collection container 29 through the 54th flow channel fc54. FIG. 28 illustrates the operation in step ST5. In step ST5, the flow rate of the rinse liquid passing through the spiral flow channel 27 becomes high, and accordingly, the operation in the step allows for the extraction of CD34 positive cells from the white blood cells contained in the rinse liquid. Thus, the CD34 positive cells are sent to the collection container 29 through the 54th flow channel fc54. Subsequently, the controller 30 controls the valves v6 and v8 to be in their closed states so as to cancel the 51st flow channel fc51 and the 54th flow channel fc54. Step ST5 is thus finished. The CD34 positive cells in the collection container 29 may be subjected to a culturing step afterward.

According to the fourth embodiment as described above, the separation apparatus 1 includes the spiral flow channel 27 which, upon delivery of the white blood cells collected from the filter 24, extracts CD34 positive cells from the delivered white blood cells. Thus, in addition to the effects discussed above, CD34 positive cells contained in white blood cells of blood can also be collected.

According to at least one embodiment or the like described above, air bubbles that would otherwise remain in the filter in the course of introducing blood into the filter can be eliminated or reduced.

The term “processor” or the like used herein refers to, for example, a central processing unit (CPU) or a graphics processing unit (GPU), or various types of circuitry which may be an application-specific integrated circuit (ASIC), a programmable logic device (such as a simple programmable logic device (SPLD), a complex programmable logic device (CPLD), or a field programmable gate array (FPGA)), and so on. If, for example, the processor is a CPU, the processor reads and executes a program or programs stored in storage circuitry (not shown in the figures) to realize the intended functions. If, for example, the processor is an ASIC, the functions are directly incorporated into circuitry of the processor in the form of a logic circuit, instead of corresponding programs being stored in storage circuitry. Each processor in the embodiments, etc. is not limited to a single circuit-type processor, and multiple independent circuits may be combined as one processor to realize the functions. Furthermore, multiple components or features as given in FIGS. 1, 10, 15 to 18, 22, and 25 may be integrated as one processor to realize their functions.

While certain embodiments have been described, these embodiments have been presented by way of example only, and are not intended to limit the scope of the inventions. Indeed, the novel embodiments described herein may be embodied in a variety of other forms. Furthermore, various omissions, substitutions, and changes in the form of the embodiments may be made without departing from the spirit of the inventions. The accompanying claims and their equivalents are intended to cover such forms or modifications as would fall within the scope and spirit of the inventions.

What is claimed is:

1. A separation apparatus comprising:
a filter configured to separate a white blood cell from blood sent from a blood container; and
a first flow channel through which the blood container and the filter are in communication with each other,
wherein the blood is introduced into the filter through the first flow channel, from below the filter, and in an anti-gravity direction.

2. The separation apparatus according to claim 1, further comprising:
a first medical liquid container for storing a wash liquid for washing the filter;
a second flow channel through which the first medical liquid container and a part of the first flow channel are in communication with each other; and
a flipping mechanism configured to flip over the filter,
wherein the flipping mechanism is configured to flip over the filter after introduction of the blood, and
the wash liquid sent through the second flow channel is introduced into the flipped-over filter through the first flow channel, from above the filter, and in a gravity direction.

3. The separation apparatus according to claim 2, further comprising:
a second medical liquid container for storing a collection liquid for collecting the white blood cell from the filter; and
a third flow channel through which the second medical liquid container and a blood outlet of the filter are in communication with each other,
wherein the collection liquid is introduced into the filter after introduction of the wash liquid, through the third flow channel and the blood outlet and in the anti-gravity direction, the blood outlet being located at a lower part of the filter.

4. The separation apparatus according to claim 3, wherein
the wash liquid and the collection liquid are same one liquid, and
the first medical liquid container and the second medical liquid container are same one container.

5. The separation apparatus according to claim 3, wherein
the wash liquid and the collection liquid are different liquids, and
the first medical liquid container and the second medical liquid container are different containers.

6. The separation apparatus according to claim 1, wherein the filter is configured to catch the white blood cell contained in the blood, while permitting a red blood cell and a platelet contained in the blood to pass through.

7. The separation apparatus according to claim 1, wherein the filter comprises a nonwoven fabric.

8. The separation apparatus according to claim 1, further comprising:
a drain container for storing a blood component that has passed through the filter; and
a suction member configured to apply a negative gas pressure to the drain container, so as to cause the blood in the blood container to be introduced into the filter through the first flow channel and to suction, toward the drain container, the blood component that has passed through the filter.

9. The separation apparatus according to claim 8, further comprising a pressurization member configured to apply a positive gas pressure to the blood container, so as to cause the blood in the blood container to be introduced into the filter through the first flow channel.

10. The separation apparatus according to claim 1, further comprising a pressurization member configured to apply a positive gas pressure to the blood container, so as to cause the blood in the blood container to be introduced into the filter through the first flow channel.

11. The separation apparatus according to claim 2, wherein the first flow channel has a length larger than a shortest distance between the blood container and the filter.

12. The separation apparatus according to claim 11, wherein the length of the first flow channel is smaller than a length which enables flipping over of the filter twice in one direction.

13. The separation apparatus according to claim 2, wherein the flipping mechanism is configured to flip over the filter by causing the filter to make a half rotation about an axis which is orthogonal to a straight line connecting a blood inlet of the filter and a blood outlet of the filter.

14. The separation apparatus according to claim 2, further comprising a holder holding the filter,
wherein the filter, the holder, the first flow channel, the first medical liquid container, and the second flow channel are accommodated in a cartridge, and
the flipping mechanism is configured to flip over the filter by pulling the holder through a part of the cartridge using magnetic force, and turning the pulled holder upside down.

15. The separation apparatus according to claim 2, further comprising:
a first valve configured to open and close the first flow channel;
a second valve configured to open and close the second flow channel; and
a controller configured to control the first valve and the second valve for opening and closing actions,
wherein the controller is configured to
control, among the first valve and the second valve, only the first valve to be in an opened state so as to introduce the blood into the filter, and
control, among the first valve and the second valve, only the second valve to be in an opened state so as to introduce the wash liquid into the filter.

16. The separation apparatus according to claim 3, further comprising a spiral flow channel configured to extract, upon delivery of the white blood cell collected from the filter, a CD34 positive cell from the white blood cell.

17. A separation method performed by a separation apparatus, the separation apparatus comprising: a filter configured to separate a white blood cell from blood sent from a blood container; and a first flow channel through which the blood container and the filter are in communication with each other,
the separation method comprising causing the blood to be introduced into the filter through the first flow channel and in an anti-gravity direction.
